(19) United States
(12) Patent Application Publication
(10) Pub. No.: US 2005/0159639 A1
(43) Pub. Date: Jul. 21, 2005

(54) Physiologically based control system and method for using the same

(76) Inventors: Mikhail Skliar, Salt Lake City, UT (US); Guruprasad A. Giridharan, Salt Lake City, UT (US)

Correspondence Address: KENNETH E. HORTON;KIRTON & MCCONKLE, 60 EAST SOUTH TEMPLE, SUITE 1800, SALTLAKE CITY, UT, 84111, US

(21) Appl. No.: 11/015,523

(22) Filed: Dec. 17, 2004

Related U.S. Application Data
(63) Continuation-in-part of application No. 10/147,259, filed on May 15, 2002.

(51) Int. Cl. A61N 1/362

(52) U.S. Cl. 600/16

(57) ABSTRACT

A device and method for maintaining a constant average pressure difference between the inlet and outlet of a pump for a body fluid, leading to an adequate flow for different pathological and physiological conditions, is described. The device and method allow for automatic adjustment of the pump operation to increase or decrease the flow rate of a body fluid to meet the physiological demand of the patient. The device and method also allow the physiological constraints on the pump to be accounted for, preventing suction and minimizing back flow of the body fluid. The device and method allow implicit synchronization of the pump with the natural regulatory mechanism for meeting patient's demand. The natural regulatory system continuously adjusts the parameters of the circulatory system to meet physiological demand for a body fluid. Maintaining constant pressure differential between the inlet and outlet of a pump, or a part of the body leads to the adaptation of the flow rate of the body fluid to physiological changes in response to the patient's clinical or physiological conditions.

### REFERENCE TO RELATED APPLICATION

This application is a continuation-in-part of U.S. patent application Ser. No. 10/147,259 (“the '259 application”), the entire disclosure of which is incorporated herein by reference.

### FIELD OF THE INVENTION

The invention relates to medical devices and methods for using such devices. Specifically, the invention related to control systems for pumps for body fluids and methods for using such controllers. Even more specifically, the invention relates to a controller for pumps that automatically regulates the pump in accordance with the physiological needs of the patient.

### BACKGROUND OF THE INVENTION

Numerous types of pumps have been designed to help various parts of the body pump liquids, including the bladder, kidneys, and brain. See, for example, U.S. Pat. Nos. 4,554,069, 4,787,886, and 6,045,496, the disclosures of which are incorporated herein by reference. The primary use of such pumps has been to pump blood for the heart of a patient. See, for example, 4,509,946, 4,683,894, 4,648,877, 4,750,868, 5,007,927, 5,599,173, 5,807,737, 5,888,242, 5,964,694, 6,082,105, 6,135,943, 6,164,920, and 6,176,822, the disclosures of which are incorporated herein by reference.

Blood pumps for assisting or replacing the heart have been—and are being developed—in a number of forms. Implantable and extracorporeal blood pumps come in two varieties: total artificial heart (TAH), which completely replaces the natural heart, and ventricular assist device (VAD), which works in parallel with the weakened natural heart. The reliability issues of a permanent mechanical cardiac replacement or assist device favor continuous flow implantable blood pumps, though pulsatile mechanical pumps are also being actively developed. The VAD has an important advantage over the TAH in that, as shown in several clinical cases, once the heavy pumping load is relieved from the natural heart, it may completely recover to a point that no further mechanical assistance is needed. Both VAD and TAH hold the potential to become a long-term alternative to donor heart transplantation, an approach known as the blood pump destination therapy.

Despite significant and continuing progress in developing better artificial blood pumps, the key problem of physiological control of continuous flow blood pumps, which allows for automatic and autonomous response to the patient's physiological cardiac demand has not been solved, and control systems for physiological control of artificial blood pumps do not exist. The flow rate generated by a continuous flow VAD, such as the DeBakey pump, is selected manually by a physician or other trained hospital personnel. Mobile patients can operate an implanted continuous flow VADs in one of two ways: “automatic” and manual. During automatic control the patient, following guidelines provided by the doctor, manually sets the desired pump rpm or flow rate depending on the level of physical activity. The VAD controller automatically adjusts the electrical current and voltage applied to the pump, to achieve and maintain the desired rpm or flow setpoint. No reliable feedback based on physiological measurements (such as pressures, flows, O₂ saturation, lactic acid concentration in blood, CO₂ pressure, etc.) is available. In manual mode, the patient directly adjusts the pump rpm by “twisting the knob” until a perceived comfort level of perfusion is achieved.

One type of controller for VADs has recently been proposed. See Waters et al. “Motor Feedback Physiological Control for a Continuous Flow VAD” Artificial Organs 1999; 23(2) 480-486, the disclosure of which is incorporated herein by reference. Waters et al. propose a Proportional-Integral (PI) control system that was developed for a simple computer model of circulatory system. The assumptions made in this work are unrealistic, including continuous flow throughout the circulatory system, no heart valves and linear correlation between pump generated pressure difference, ΔP, and pump voltage, current, and rpm. As such, this proposed controller is not suitable to be used in patients and a more suitable—and realistic—type of controller needs to be developed.

### SUMMARY OF THE INVENTION

The invention provides a control system—including a controller—for continuous and pulsatile flow body fluid pumps that automatically responds to physiological demand. The invention includes a feedback controller designed to maintain physiologically sufficient flow of the needed body fluid. The invention operates by automatically adjusting parameters of the mechanical pump so that some key pressure difference or pressure differences in the circulatory system are maintained close to the reference value.

In the case of the left ventricular assist devices, examples of such differential pressures include an average or instantaneous pressure difference between the left heart and aorta; a constant instantaneous or time averaged pressure difference between the inlet and outlet of the pump; and an average or instantaneous pressure difference between pulmonary venous and aortic pressures. Maintaining one of the listed pressure differences will result in indirect control of other pressure differences in circulatory system, and will affect the body fluid flow in the system. The fluid flow is strongly dependent on the resistance of the circulatory system, controlled by natural regulatory mechanisms, with an appropriately selected pressure differences remaining nearly constant over broad range of physiological and clinical conditions.

The invention, by manipulating the artificial pump to maintain the selected reference pressure difference, ensures the adequate flow of the body fluid for different physiological and pathological conditions as long as the natural regulation continues to adequately function by adjust circulatory resistance. The invention allows automatic adjustment of the pump parameters via a control system to maintain the reference pressure difference, thereby preventing suction and minimizing back flow of the body fluid. The control system relies on implicit synchronization of the pump with the natural regulatory mechanisms and thus can be continually and automatically adjusted to an optimal level in response to the patient's physiological condition.

### BRIEF DESCRIPTION OF THE DRAWINGS

FIGS. 1-36 are views of several aspects of the control systems and methods for using the same according to the invention, in which:

FIGS. 1-5 illustrate a model used in the fluid system in one aspect of the invention;

FIGS. 6-7 illustrate the conditions of a healthy heart in one aspect of the invention;

FIGS. 8-9 illustrate the conditions of a weakened heart in one aspect of the invention;

FIGS. 10-11 illustrate the conditions of a fibrillating, tachycardic or asystolic heart in one aspect of the invention;

FIGS. 12-13 illustrate the conditions of the circulatory system when a healthy heart is assisted with the pump in one aspect of the invention;

FIG. 14 depicts one aspect of the pump when assisting a healthy heart;

FIGS. 15-16 illustrate the conditions of a weakened heart assisted with the pump in one aspect of the invention;

FIG. 17 depicts one aspect of the pump when assisting a weakened heart;

FIGS. 18-19 illustrate the conditions of an asystolic heart assisted with the pump in one aspect of the invention;

FIG. 20 depicts one aspect of the pump when assisting an asystolic heart;

FIGS. 21-22 illustrate the conditions of a healthy heart during exercise when assisted with the pump in one aspect of the invention;

FIG. 23 depicts one aspect of the pump when assisting a healthy heart during exercise;

FIGS. 24-25 illustrate the conditions of a weakened heart during exercise when assisted with the pump in one aspect of the invention;

FIG. 26 depicts one aspect of the pump when assisting a weakened heart during exercise;

FIGS. 27-28 illustrate the conditions of an asystolic heart during exercise when assisted with the pump in one aspect of the invention;

FIG. 29 depicts one aspect of the pump when assisting a asystolic heart during exercise;

FIG. 30 shows the one possible schematic in one aspect of the invention;

FIG. 31 shows typical operating characteristics of the pump and the circulatory system in one aspect of the invention;

FIG. 32 shows the baseline parameters of the circulatory system used in simulating the human equivalence of a normal, failing and asystolic LV in one aspect of the invention;

FIG. 33 compares operating characteristics of the pump and circulatory system in one aspect of the invention;

FIG. 34 depicts the schematic of the circulatory system with the assist device used in testing one aspect of the invention;

FIG. 35 compares the total flow generated with ΔPa, ΔP, and constant rpm strategies with and without the VAD in (a) normal LV, (b) failing LV and (c) asystolic LV; and

FIG. 36 shows the pressure volume (PV) loops with and without VAD assistance using constant rpm, ΔP, and ΔPa control strategies under the following test conditions: (a) normal LV during rest; (b) normal LV during exercise; (c) failing LV during rest; and (d) failing LV during exercise.

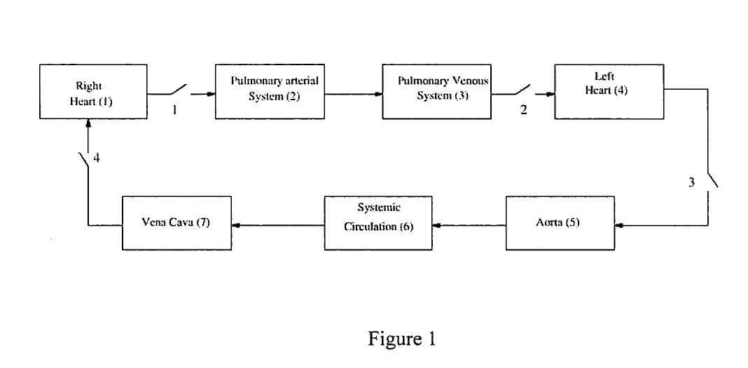

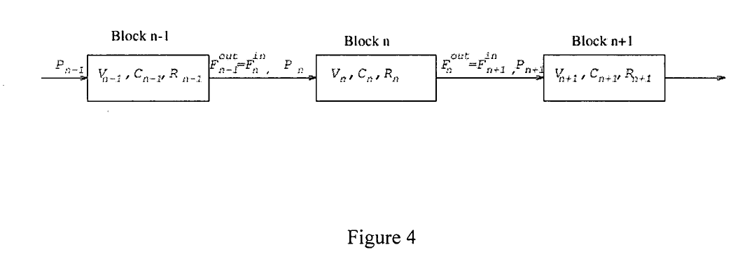

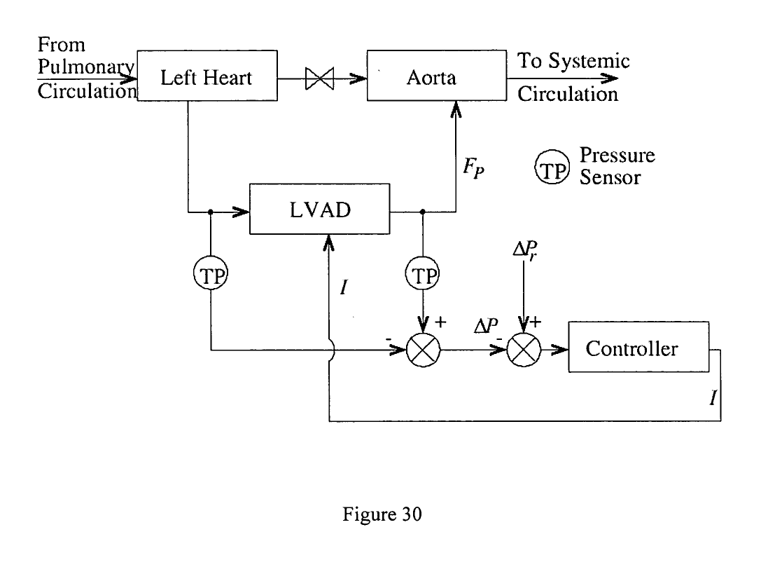

FIGS. 1-36 illustrate specific aspects of the invention and are a part of the specification. Together with the following description, the Figures demonstrate and explain the principles of the invention and are views of only particular—rather than complete—portions of the invention.

### DETAILED DESCRIPTION OF THE INVENTION

The following description provides specific details in order to provide a thorough understanding of the invention. The skilled artisan, however, would understand that the invention could be practiced without employing these specific details. Indeed, the present invention can be practiced by modifying the illustrated system and method and can be used in conjunction with apparatus and techniques conventionally used in the industry. For example, the invention is described below for pumps used in pumping blood for the heart, but could be modified for other body fluids and other body parts that pump liquids, including the bladder, kidneys, heart-lung machines and intravascular blood pumps.

The invention includes a fluid system for pumping body fluids and method for using the same, including the systems and methods described in Giridharan et al., Physiological Control of Rotary Blood Pumps: An In Vitro Study, ASAIO Journal 2004, pp. 403-409, and Giridharan et al., Modeling and Control of a Brushless DC Axial Flow Ventricular Assist Device, ASAIO Journal, 2002, pp. 272-289, the disclosures of which is incorporated herein by reference. The fluid system of the invention comprises a pump system, a control system for controlling the pump system, and any other necessary components for the fluid system to operate. The pump system contains a pump and any devices associated with operating the pump. Examples of pumps include both continuous and non-continuous flow pumps known in the art. The control system contains a controller for controlling or regulating the pumping system and any other devices associated with regulating the pump. Examples of controllers that can be used in the invention are described below.

The fluid system of the invention is used, for example, to pump body fluids in a controlled manner. In one aspect of the invention, the fluid system of the invention is used to pump blood through the circulatory system. In this aspect of the invention, the pumping system contains any blood pump that is conventionally used, e.g., a VAD. In this aspect of the invention, the pumping system also contains components or devices—such as tubing, connectors, valves, sensors, power supply, and/or the like—that are typically used with the blood pump during its operation. See, for example, the description of such components or devices in U.S. Pat. Nos. 5,888,242, 4,683,894, 4,509,946, 4,787,886, 5,007,927, 5,599,173, 5,807,737, and 6,045,496, as well as European Patent Application No. 0503839A2, the disclosures of which are incorporated herein by reference.

In this aspect of the invention, the control system contains any suitable controllers that functions to regulate or control the pumping system and the pump. In this aspect of the invention, the control system also contains components or devices—such as sensors, a monitoring system, and/or a feedback system—that are typically used with the controller during its operation. See, for example, the description of such components or devices in U.S. Pat. Nos. 5,888,242, 4,683,894, 4,509,946, 4,787,886, 5,007,927, 5,599,173, 5,807,737, and 6,045,496, as well as European Patent Application No. 0503839A2, the disclosures of which are incorporated herein by reference.

In one aspect of the invention, two pressure sensors and an rpm sensor were used to control the pump. In another aspect of the invention, however, the pressure sensors can be eliminated by using readily available measurements of the pump rpm, voltage and current information to estimate the pressure differential. Eliminating the sensors leads to a controller that estimates the pressure difference using the intrinsic pump parameters to control the VAD, eliminating the need for failure-prone pressure sensors and resulting in a more reliable control system.

In one aspect of the invention, the objective of the control system for the blood pump is to maintain the pressure difference between the left heart (LH) (and more specifically the left ventricle) and the aorta close to the specified reference pressure difference, ΔP. The body maintains an approximately constant average ΔP and varies the vascular resistance to maintain the required pressure and flow of blood. Maintaining the ΔP at the desired level synchronizes the assist and natural perfusion, thereby incorporating natural cardiovascular regulation into the controller and allowing for simple control.

In another aspect of the invention, the objective of the control system for the blood pump is to maintain the pressure difference between the pulmonary vein (or the left atrium) and the aortic pressure close to the specified reference ΔP. Maintaining the pressure differences difference between the pulmonary vein or left atrium and the aortic pressure will indirectly control the pressure difference between left ventricle and aorta, the pressure difference across the VAD, and other key pressure differences in the circulatory system, as well as affect the body fluid flow in the system. Selecting the control objective is one aspect of the invention, in which a key pressure differences in the circulatory system are maintained with mechanical blood pumps, while relying on the natural regulation to adjust the resistances to the blood flow to meet the physiological demand and changing cardiac function.

Another aspect of the invention is used for pulsatile ventricular assist devices, as well as for continuous-flow and pulsatile TAH. For the total artificial heart, the blood pump should be controlled to maintain key pressure differences at the average reference values. In the case of pulmonary circulation, this difference is between pulmonary arterial (or right atrial) and vena cava pressures. In the case of systemic circulation, this difference is between pulmonary venous (or left atrial) and aortic pressures. Maintaining key pressure differences and relying on the natural regulatory mechanisms to adjust the vascular resistance in response to changing cardiac demand can result in the physiological perfusion achieved with the TAH.

The controller used in the invention can be any controller that functions to control that pump under the conditions described herein. In one aspect of the invention, the controller can minimize the difference between the reference and the actual differential pressure when changes occur in the portion of the circulatory system being controlled. Thus, any controller obtaining that function can be used in the invention.

In one aspect of the invention, the controller will be able to work with various types of pumps in the pumping system. As noted above, the pumps that can be used in the invention include, for example, both continuous flow pumps and non-continuous flow (e.g., pulse or pulsatile) pumps. Thus, the controller should be selected to, where appropriate, work with these types of pumps.

In one aspect of the invention, the controller can have additional functions than merely controlling the pressure differential as described above. For example, the controller could also have functions such as: monitoring the pump operation for faults and other abnormal operations caused by mechanical, electrical or other failures of any component necessary for the operation of an artificial pump; detecting the changes in clinical status of the patient, such as hypertension, hypotension and internal bleeding; and automatically responding to the detected abnormal operation, including sounding an alarm, notifying human operators, and automatic drug measurement and delivery.

In one aspect of the invention, such as where the invention is used for controlling the pressure between the LH and the aorta, a proportional-integral (PI) controller was designed to vary the motor current of the blood pump to minimize the difference between the reference and the actual differential pressure when changes occur. One normal case and two different pathological cases (second and third cases) were then simulated to test the controller operation. In the first case, the VAD was attached to a normal healthy heart (as is the case following the recovery of natural left ventricle function or during testing with animals). In the second case, the VAD was attached to a weakened left heart. And in the third case, the VAD was attached to an asystolic left heart.

The design of the controller is broken into the steps of selecting the control objectives, selecting the measurements (or control inputs) to be used in the feedback, and then designing the control algorithms. The design process is iterative in nature, with the each design step followed by performance evaluation that motivates the re-design goals. Developing a model of the pump-assisted circulatory system is useful during the iterative process of controller design.

When selecting an adequate model of the pump-assisted circulatory system for design of the pump controller, it is important to avoid overwhelming complexity of the full-scale model of the entire circulation system. At the same time, however, all relevant characteristics needed for the controller design must be retained. The model used to design the invention preserves such characteristics as nonlinearity, pulsatility, and discontinuity due to the effects of the natural heart valves.

The model for the PI controller design combines the circulation model (the model of the circulatory system being controlled) with a model of a continuous flow VAD. The model subdivides the circulatory system into an arbitrary number of lumped parameter blocks (or elements), each characterized by its own resistance, compliance, pressure and volume of blood. In one aspect of the invention, the model has eleven elements as illustrated in FIG. 1: 4 heart valves (1, 2, 3, 4), and 7 blocks including left heart (LH), right heart (RH), pulmonary and systemic circulation, vena cava and aorta. Hemodynamic details (such as velocity profile) are not incorporated into this model, but can be if desired.

The detail of the model can be varied, e.g., increased or decreased. The detail can be increased by adding additional elements or by increasing the number of elements (or blocks). For example, the detail can be increased by subdividing the pulmonary and systemic circulations into constituent sub-elements. As well, the detail can be decreased by removing some of the elements or by reducing the number of elements by combining blocks together.

Operation of each elements/block depends on its resistance (R) to the blood (or other body fluid) flow (F) and its compliance (C), which quantifies the ability of a given block to store a given blood (or other body fluid) volume (V). Two idealized elements, resistance and storage, are used to characterize each block. The storage element provides zero resistance to the flow, whereas the resistive element has zero volume. The resistance of an element or block is a function of the pressure drop and the blood flow across the block. The flow rate in and out of any block is a function of the pressure drop and resistance. The compliance is a function of the pressure and the stored volume of blood.

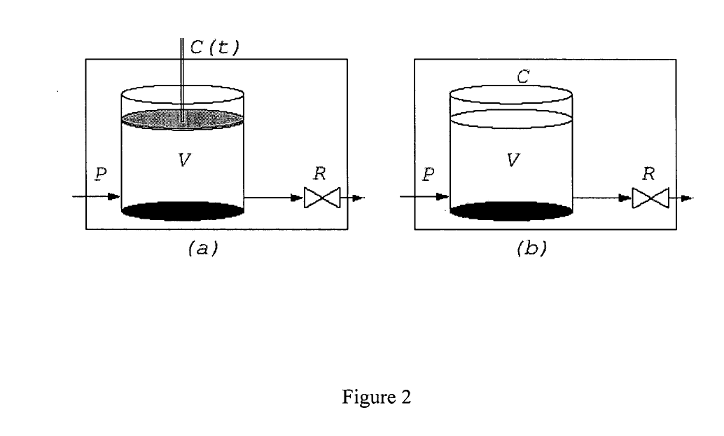

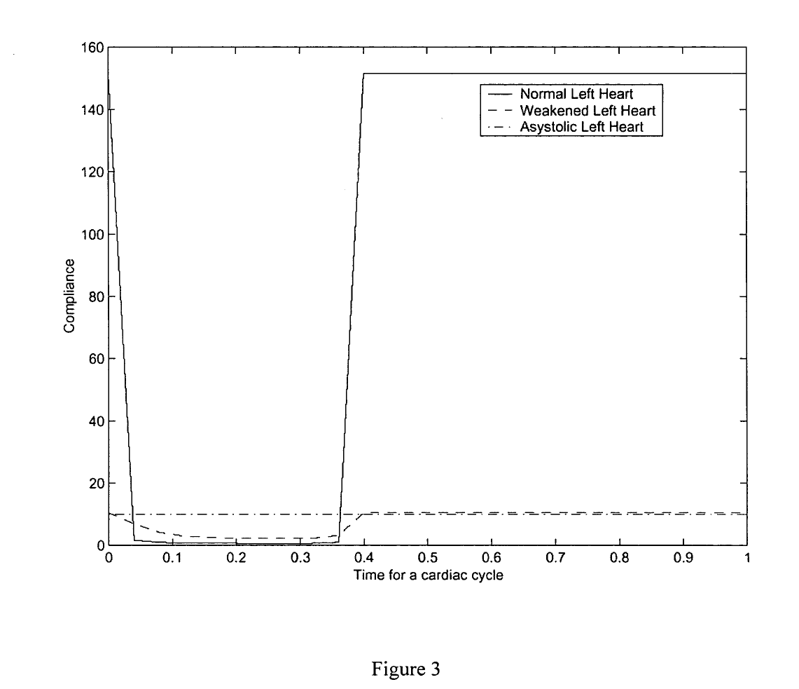

As illustrated in FIG. 2, each block can be categorized as passive (FIG. 2(a)) or active (FIG. 2(b)). Active blocks represent heart chambers and are characterized by the varying compliance within each cardiac cycle. The rest of the blocks are passive. The varying compliance of the active blocks is responsible for the progression of the heartbeat. FIG. 3 illustrates example values of the compliance of an active block.

The volume of blood in any given block can be estimated using a macroscopic material balance for that block. Accordingly, the volume of fluid for that block is a function of the resistance and compliance, which will differ in different patients and under different pathological conditions. In the invention, typical C and R values were assumed for all passive and active blocks and then were adjusted to reflect different pathological conditions under the three different cases described above.

The model includes four heart valves depicted in FIG. 1 as switches. A valve can be either fully open or fully closed. A valve is in open position when the upstream pressure is greater than the downstream pressure and is otherwise closed to prevent the back flow. In an open position, each valve has a finite and constant resistance to the blood flow. The resistance becomes infinite in the closed position. In other words, the heart valve is modeled as a block with no storage and with resistance that takes finite or infinite value depending on the sign of the differential pressure.

The model of the circulatory system is, therefore, a hybrid system that includes both dynamic and logical components. This circulatory model can be modified to include any desired pump and to evaluate the performance of the assisted circulatory system with the designed controller. In one aspect of the invention, the circulatory model is modified for an axial flow LVAD as a pump. In this aspect of the invention, a brushless DC motor drives the VAD. A typical brushless DC motor is described in U.S. Pat. No. 5,888,242, the disclosure of which is incorporated herein by reference.

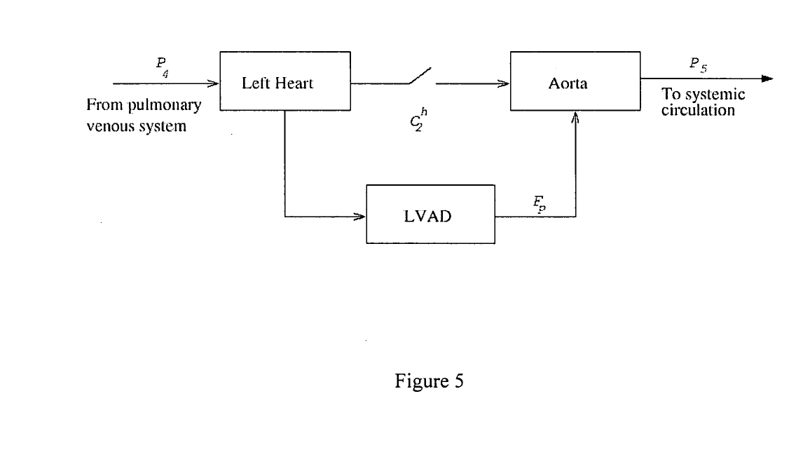

This model and controller can be designed to be used in parallel or in series with the heart. In one aspect of the invention, the model and controller were designed for the case when the assist device works in parallel with the heart as depicted in FIG. 5. In this instance, the integration of the circulatory and LVAD models is simple and only affects the left heart and the aorta blocks.

The rpm sensor can optionally be integrated into the VAD design, as is the case with the DeBakey pump. However, measuring the differential pressure requires detecting the inlet and outlet pressures. In one aspect of the invention, two pressure sensors can be implanted for such detection. In another aspect of the invention, a sensorless VAD control system estimates the pressure differential by measuring the pump current I, voltage V, and rotational speed ω, and using the model of the VAD during the estimation. See, for example, U.S. Pat. No. 6,135,944, the disclosure of which is incorporated herein by reference.

In the model, the compliances and resistances typically differ from patient to patient, and variations can occur for any given patient over any given time period. Adaptive control strategies, as known in the art, can be used to account for inter- and intra-patient variability in the circulatory system for better control.

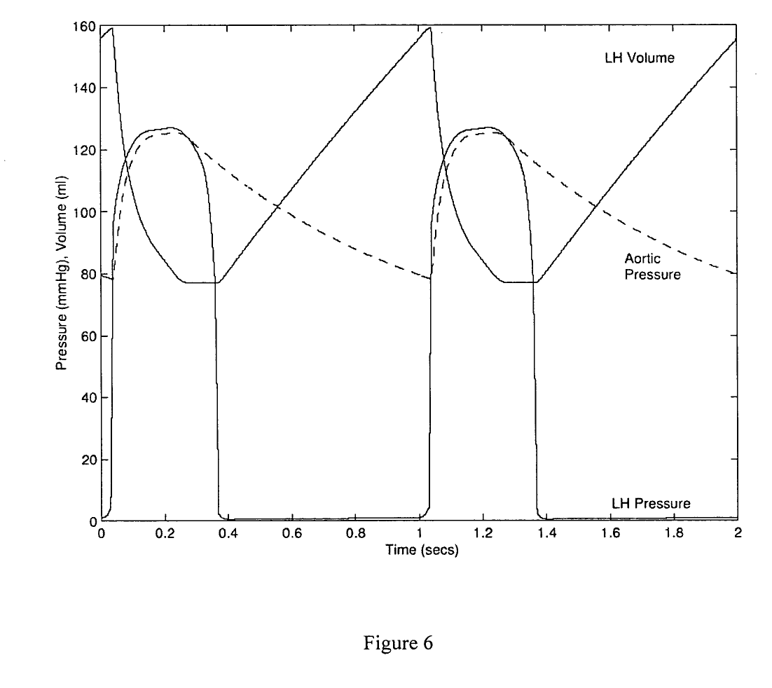

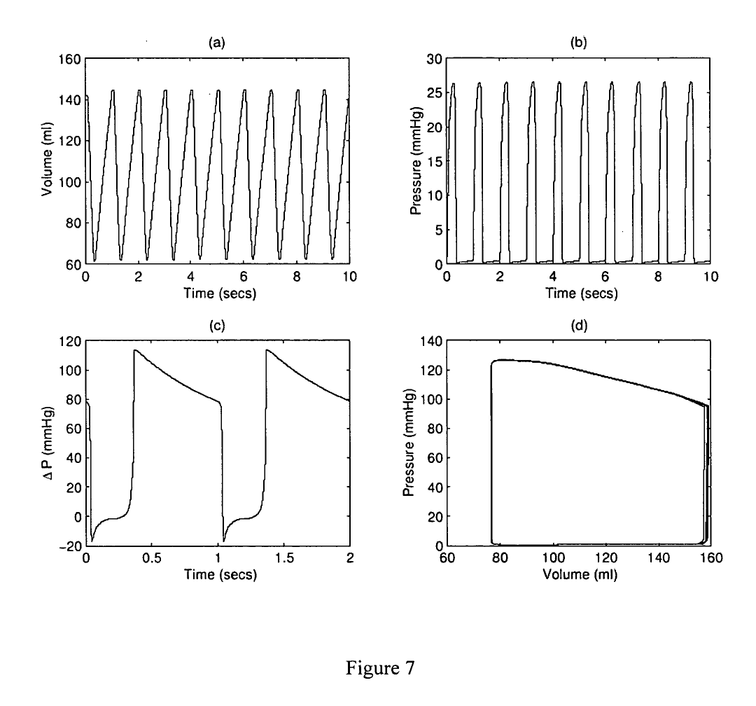

The performance of the designed controller described above, was tested under three different cases with different clinical conditions simulated. The first case is the typical healthy heart whose characteristics are depicted in FIGS. 6 and 7. The stroke volume, which is the difference between the maximum and minimum volumes in the cardiac cycle, is 80 ml. The aortic systolic and diastolic pressures are 125/80 mmHg. There is a flow between the left heart (LH) and the aorta when the aortic pressure is lesser than the LH pressure. FIG. 7a shows the stroke volume of the right heart (RH) to be approximately 80 ml, the same as the left heart. The RH peak pressure is 27 mmHg as seen from FIG. 7b. The normal range for RH pressure is 23-35 mmHg. The pressure difference between the aorta and the LH with progression of time is shown in FIG. 7c. The work done per stroke of the heart can be calculated using the area enclosed by the pressure-volume loop as illustrated in FIG. 7d.

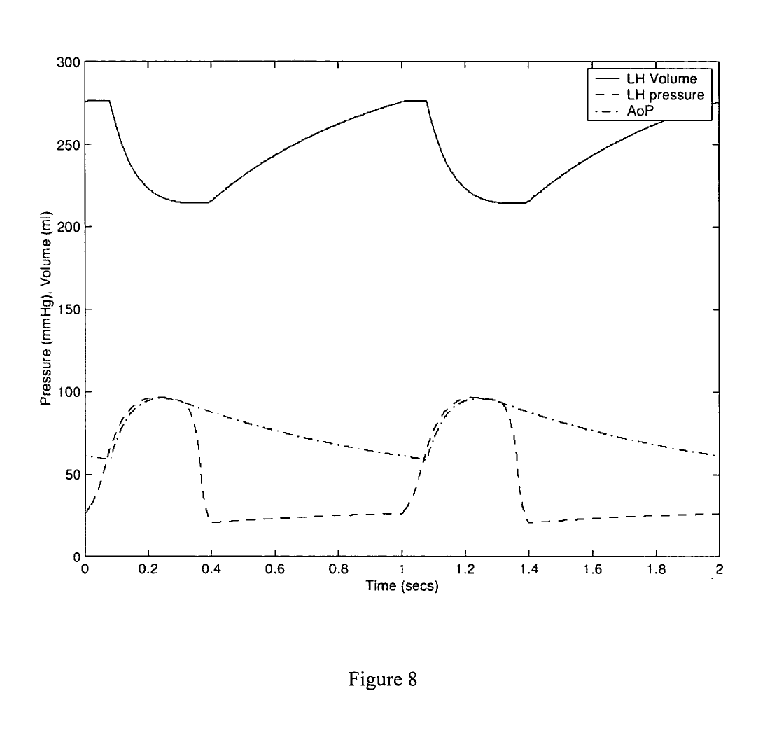

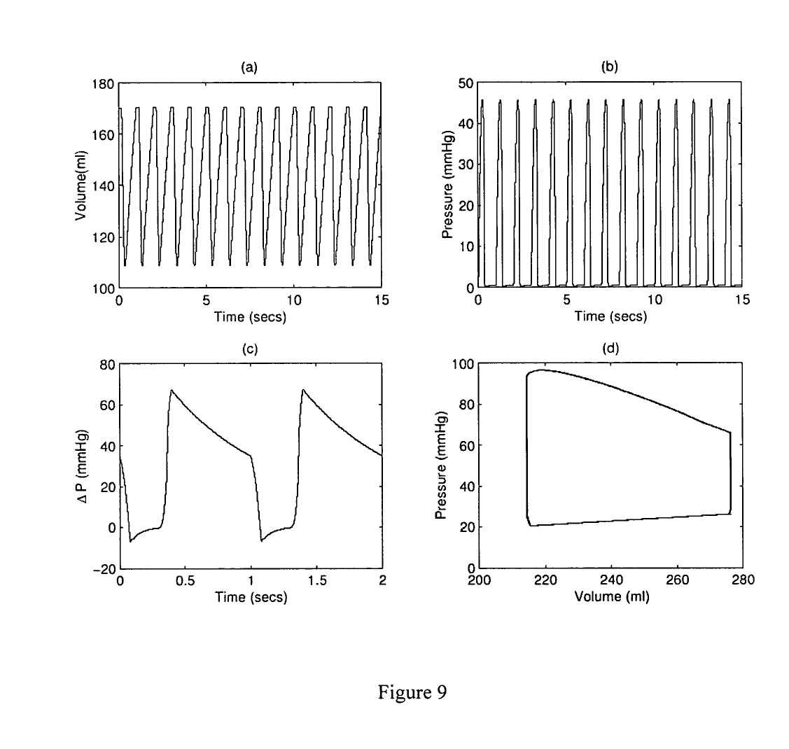

The second case is the failing heart whose characteristics are depicted in FIGS. 8 and 9. The failing heart has a lower stoke volume of approximately 60 ml and the aortic systolic and diastolic pressures are around 95/60 mmHg. A comparison with FIG. 6 shows that the LH volume is considerably higher than normal. The RH pressure is also much higher at 45 mmHg as depicted in FIG. 9b, which is typical for RH pressure with a failing left heart. Though not shown in figures, the simulation predicts edema in the pulmonary circulation, in the failing heart case. FIG. 9d shows that the work done by the weakened heart is less than the work done by the healthy heart, as the area of the pressure-volume loop is less than that of the normal heart.

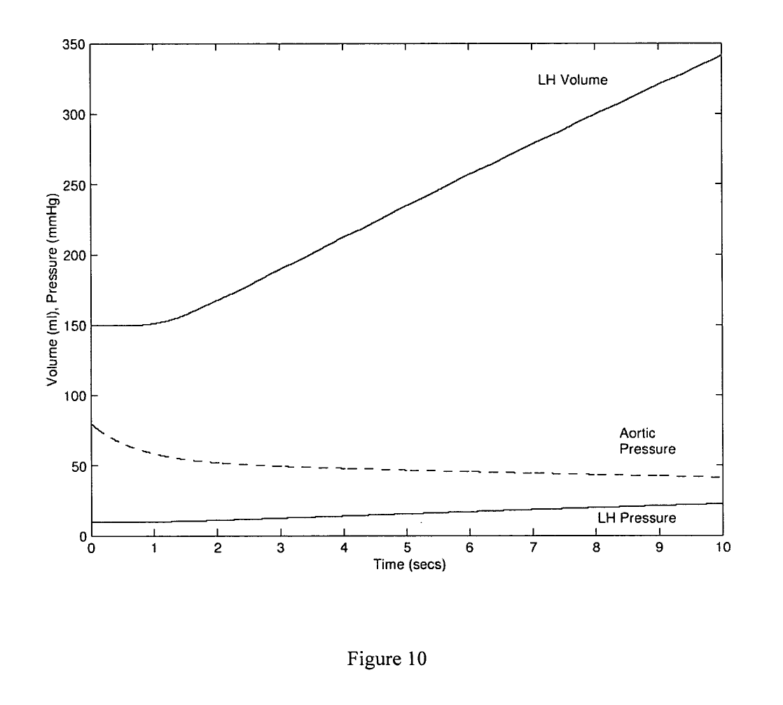

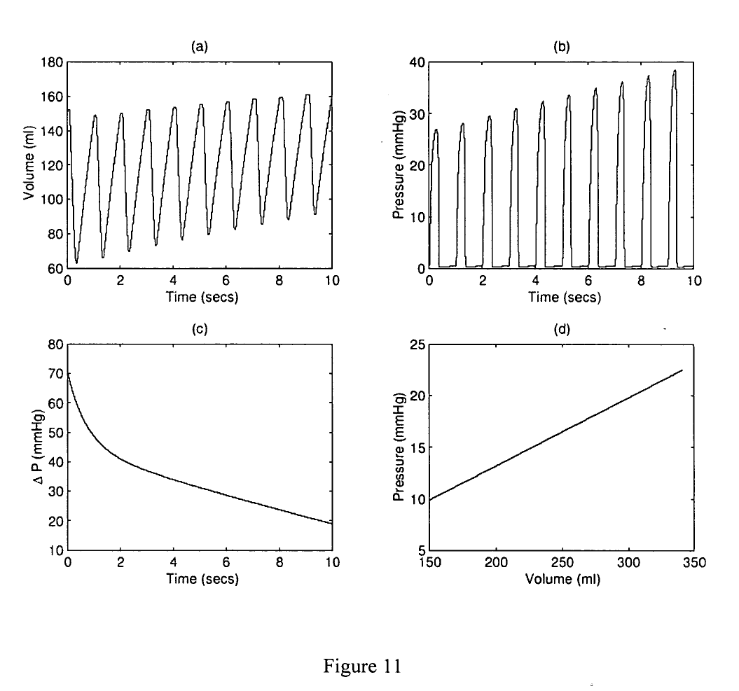

The third case is the asystolic LH heart whose characteristics are depicted in FIGS. 10 and 11. Asystole occurs to the whole heart, i.e. both LH and RH. The asystolic LH is used as an artifact to test the effectiveness of the PI controller. The LH volume should not rise above a certain value as the compliance for an asystolic LH heart decreases rapidly with increase in volume above a certain value. A constant compliance was assumed for the left heart for all LH volumes, resulting in the volume increase until about 1000 ml is reached when the physical forces equilibrate. This artifact does not affect the subsequent simulation with VAD feedback control since the controller is designed to keep the volume within narrow bounds, justifying the assumption of constant LH compliance. FIGS. 11a and 11b show an increasing RH volume and pressure. The absence of the pressure-volume loop in FIG. 11d indicates the terminal condition as the native heart produces no working stroke. FIG. 11c shows ΔP reducing constantly, indicating a sharp and definite decrease in circulation of blood. An asystolic left heart is the worst case for the LVAD load as it has to do all the work.

In one aspect of the invention, the controller should function automatically. Further, the controller should function to adapt the VAD generated flow to the changing physiological requirements of the patient. Any controller (and control system) operating under these conditions can be used in the invention.

In one aspect of the invention, the controller maintains a constant instantaneous or time average pressure differential between the inlet and outlet of the pump, denoted as ΔP. Maintaining a this pressure differential is an effective way to the correct adaptation of the cardiac output to the changing requirements of the body because it is known that the vascular bed resistance can increase or decrease by a factor of 2 to 5. Since the blood flow is directly proportional to ΔP and inversely proportional to the vascular bed resistance, maintaining a constant ΔP with changing bed resistance can increase or decrease the flow rate by a factor of 2 to 5. However, different pressure differences in the circulatory system may be controlled, which will simultaneously, though indirectly control ΔP.

The reference ΔP can be maintained by adjusting the pump rpm. The pump rpm should be adjusted within physiologically admissible limits despite changing patient's vascular resistance, stroke volume, and pulse of the natural heart. All of these factors represent—as known in the art—the response to natural regulatory mechanisms to the changing physiological cardiac output demands. By maintaining the prescribed ΔP, the assist and natural perfusion can be synchronized, indirectly incorporating natural cardiovascular regulation into the VAD control. Controlling ΔP may be accomplished with simple control algorithms. Further, basing the control system on controlling ΔP minimizes the number of components in control system: it requires implanting only pressure sensors or using a control system where ΔP is estimated from the readily measurable characteristics of pump itself, such as voltage, current, and rpm.

An additional advantage for selecting ΔP as a feedback for the control system is that controlling ΔP can be used to ensure that the pump rpm is maintained within the physiological limitations. One extreme—collapse of the heart—stablishes the physiological limit on the minimal volume of blood in the heart chamber, and can be translated into the constraints on the pump rotational speed as a function of blood volume. The back-flow to the heart—the other extreme—can be determined when the pump rotational speed drops below the lower limit, which depends on the vascular resistance and the varying compliance of the natural heart.

The control system of the invention also includes a feedback system that regulates the pump rpm within physiologically acceptable constraints. The feedback system minimizes the difference between the reference and the actual ΔP. Since pulsing of the heart leads to periodic changes in ΔP, and hence the rpm, the control system also functions to keep oscillations of the pump rpm low, increasing the pump life and the comfort level of the patient. To maintain the reference differential pressure, the controller manipulates the parameters of the pump operation, such as motor current and voltage of the pump. For example, the controller may manipulate the motor current until the desired trade-off between the speed of response and the rpm oscillations can be obtained.

The invention allows the design of the controllers for ventricular assist devices and total artificial heart that operate within the physiological constraints of the circulatory system and providing an adequate blood flow for a wide range of clinical conditions and exercise levels. The invention leads to an adequate perfusion with LVAD in three different cases mentioned above: healthy heart, weakened heart, and left heart asystole, under conditions of different exercise levels. The adequate perfusion was confirmed using computer simulations and laboratory experiments with mock circulatory system with different types of ventricular assist devices, including axial flow and centrifugal VADs. The controller designed to maintain ΔP, or other suitably selected pressure difference in the circulatory system, resulted in an adequate perfusion over the broad range of clinical conditions and exercise levels.

Using the control strategy of the invention, both the natural heart and the assist device can contribute to the pumping action of maintaining an average ΔP. If cardiac function improves, the native heart will increase its contribution to maintaining the reference pressure difference, with the VAD controller autonomously and automatically responding to the decreased need for assisted perfusion. Therefore, the proposed approach is well suited to the application of the ventricular assist devices in reversal-therapy of end stage heart failure (alone or in combination therapy), or as a destination therapy.

The aspect of the invention of maintaining key pressure differences with mechanical blood pumps, while relying on the natural regulation to adjust the resistances to the blood flow to meet the physiological demand, is also applicable to the case of pulsatile ventricular assist devices, as well as continuous-flow and pulsatile TAH. With the total artificial heart, the blood pump should be controlled to maintain key pressure differences at the average reference values, which, in the case of pulmonary circulation, is the difference between pulmonary arterial and vena cava pressures, and the difference between pulmonary venous and aortic pressures in the case of systemic circulation.

In one aspect of the invention, the model of the circulatory system is selected using the parameters described above. The model is used, in part, to design the controller. And then the controller is used for regulating the pressure differential between the LH and the aorta. This process could be also repeated for other circulatory systems, e.g., across the pulmonary vein, atrium, and aorta, across the total heart, between the pulmonary artery and the aorta, and even parts of the circulatory system not containing a part of the heart.

The invention can be used in series or in parallel with the desired part of the circulatory system. When in parallel, the pumping function is split between the portion of the circulatory system and the invention, e.g., both contribute some work to the pumping function. When in series, the full load of the pumping function can be carried by the invention or the circulatory system depending on whether the controller turns the pump on or off.

In another aspect of the invention, the invention can be used to control the pressure differential not only across the pump, but also across the portion of the circulatory system. For example, a constant average pressure difference can be maintained across the heart. The pump takes up the load the heart is unable to provide to maintain the required pressure difference. In case of a healthy heart, the pump takes up a negligible amount of load and in case of a severely failing heart, the pump takes up almost the entire load required to maintain the prescribed pressure difference. The invention can be demonstrated by the following non-limiting Examples.

### EXAMPLE 1

The invented method for physiological control of blood pumps was tested with a LVAD under widely varying physiological conditions. The pulse rate was 60 beats per minute during rest, and 135 bpm during exercise. The LVAD parameters used in the simulation were the same as in Choi et al. “Modeling and Identification of an Axial flow Blood Pump” Proceedings of the 1997 American Control Conference 3714-3715 (June 1997), the disclosure of which is incorporated herein by reference.

Before t=0, an unassisted perfusion was simulated. At time t=0, arbitrarily selected as the end of the diastole, the LVAD assistance was initiated with the reference differential pressure of 75 mmHg sent to the PI controller, implementing the invention in this example. The initial flow rate and rpm were set to zero, causing a large initial back flow of blood.

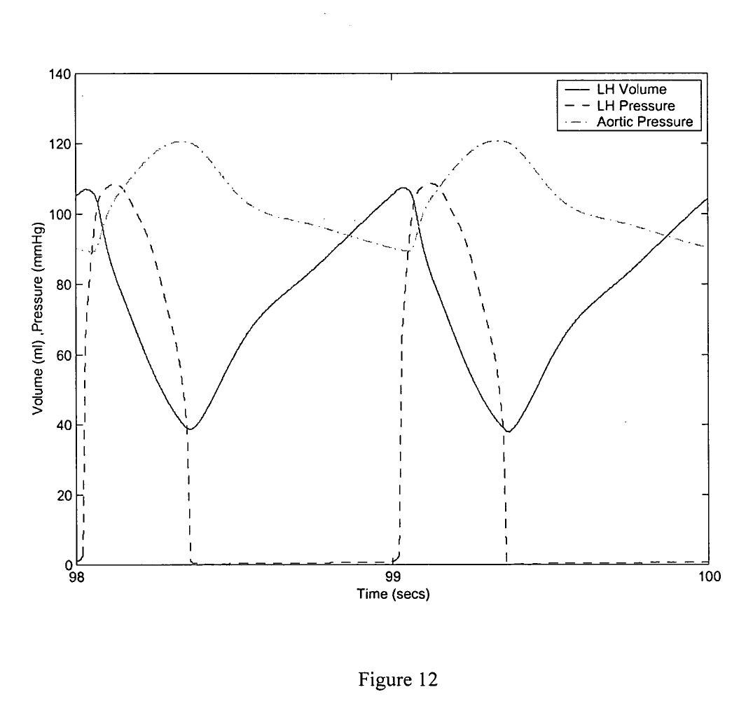

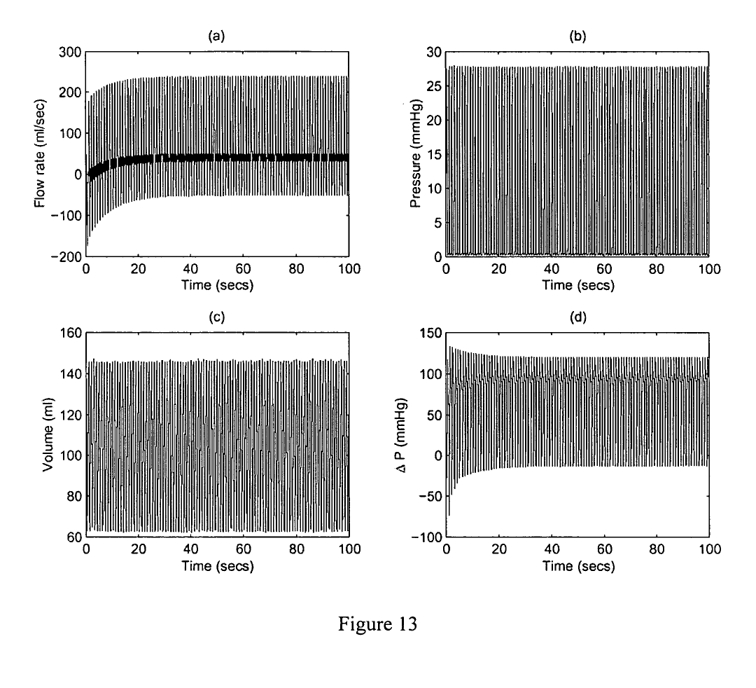

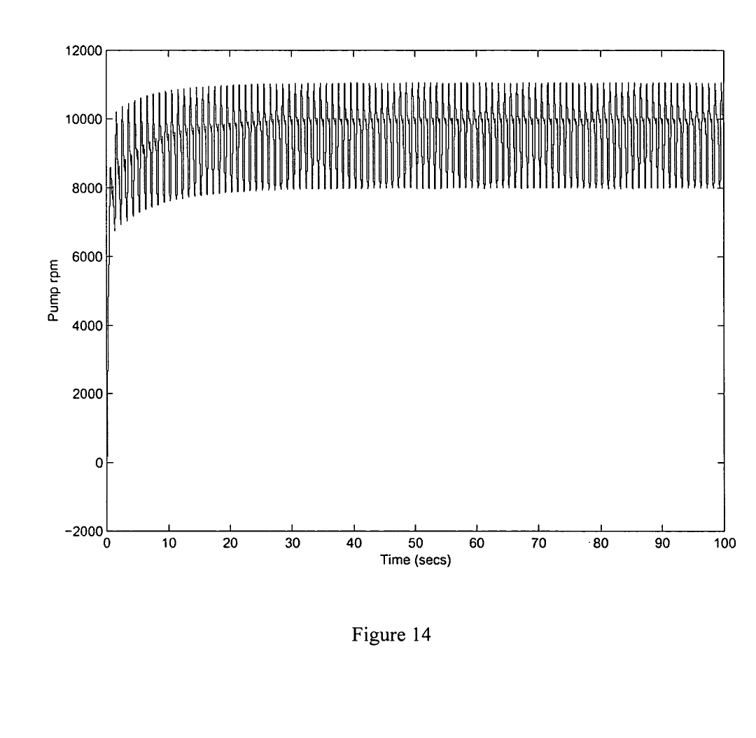

The LVAD and PI controller were first tested assuming healthy heart. FIGS. 12, 13, and 14 show results for the healthy heart with VAD assistance. FIG. 12 indicates the reduction of the LH volume from about 70/150 ml observed without VAD to about 50/80 ml during LVAD operation. The minimum volume of 50 ml gave an adequate safety margin against suction of the LH. The aortic pressure was 121/89 mmHg with low pulsatility and the LH pressure changes from 0 to about 100 mmHg. As illustrated in FIG. 13a, the pump flow rate reached the limit cycle in approximately 30 seconds. FIG. 13c shows almost the same stroke volume of 83 ml for the RH during the entire time. FIG. 13b shows the RH pressure, the maximum value of which is around 28 mmHg, well within the normal range. As depicted in FIG. 14, the rpm variations are reduced considerably after the initial transient period.

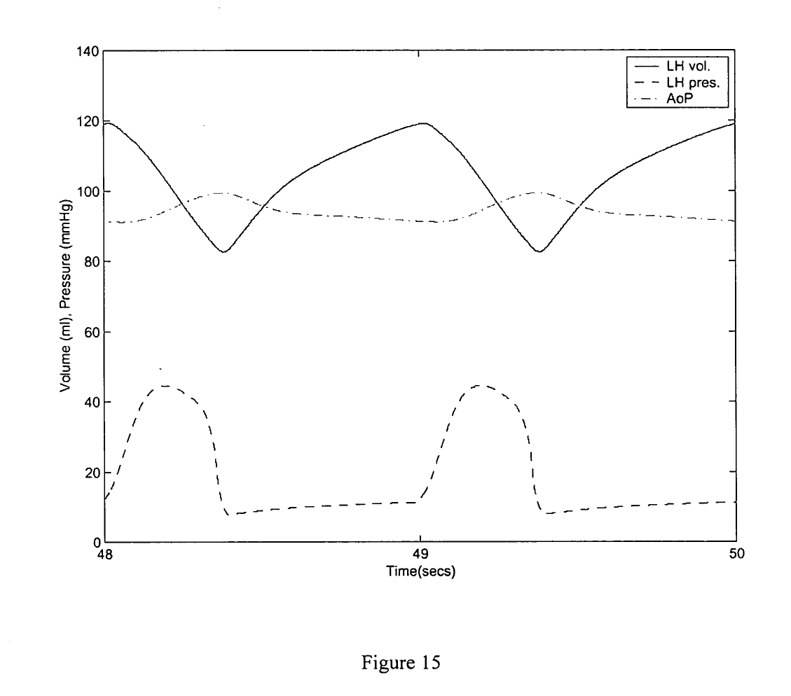

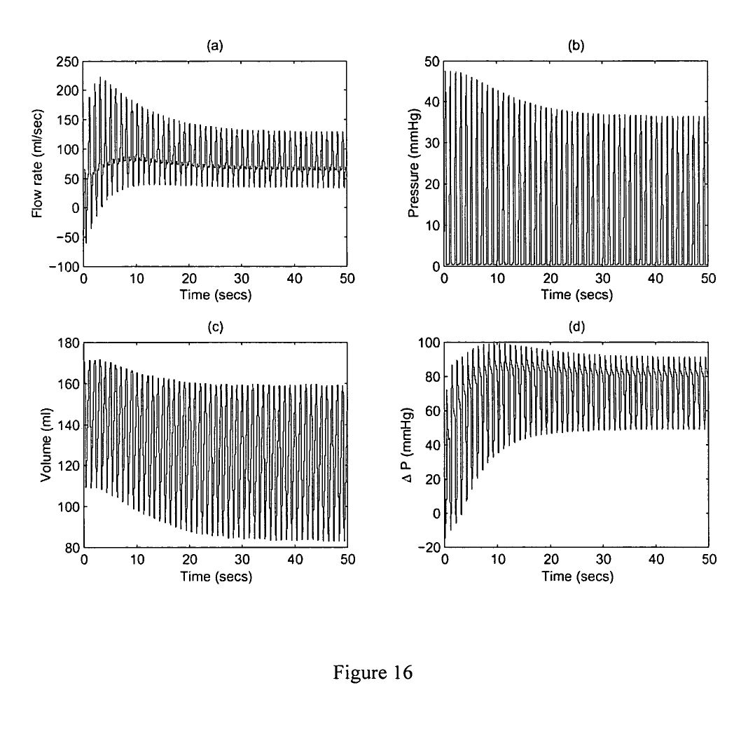

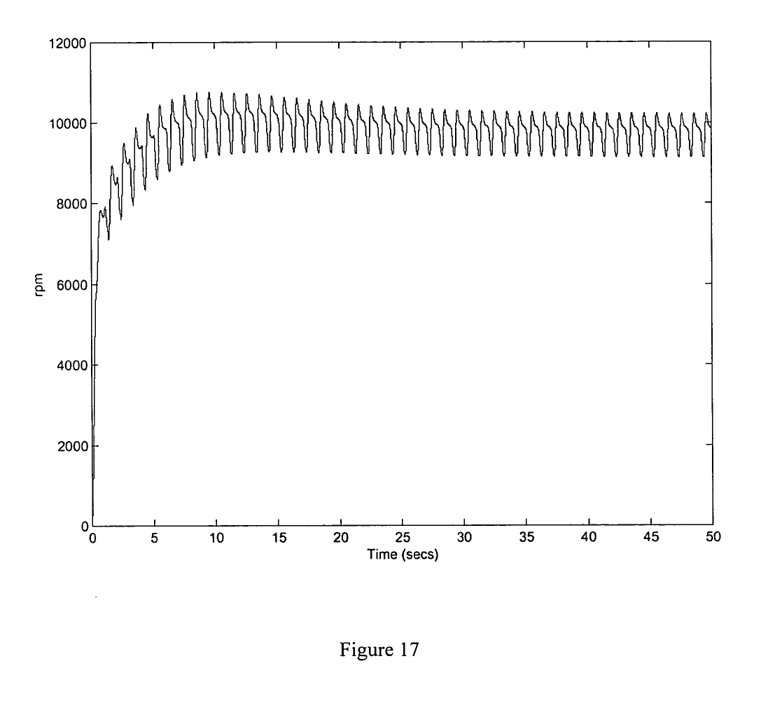

FIGS. 15, 16 and 17 show the results of the simulation for the weakened heart assisted by a VAD with the designed controller. FIG. 15 indicates a fairly constant aortic pressure around 99/91 mmHg. The LH systolic and diastolic pressures are much closer to each other compared to a healthy heart with the LVAD. The volume of the LH with VAD support reduces from 215/275 ml, observed without VAD assistance (FIG. 8), to approximately 82/119 ml, which is in the normal range. The LH pressure is reduced to about 45/11 mmHg. The RH pressure reduces to around 36/0 mmHg, FIG. 16b, which is within the normal range. The lung edema would also gradually reduce, indicating an adequate perfusion. FIG. 16a shows that there is no backflow through the pump and also that the average pressure head is close to 75 mmHg setpoint, FIG. 16d, compared to a weakened heart without a VAD. It can be noted from FIG. 16c that the stroke volume increases from 60 ml, a failure condition, to nearly 80 ml which is stroke volume for a normal heart as seen from FIG. 7. FIG. 17 shows that the rpm variations at the limit cycle are reduced and are less than rpm variations with a healthy heart, as expected since the weakened heart is unable to produce the high pressure variations that are produced by a normal heart. The initial back flow of blood illustrated in FIG. 16 is due to the zero rpm starting condition.

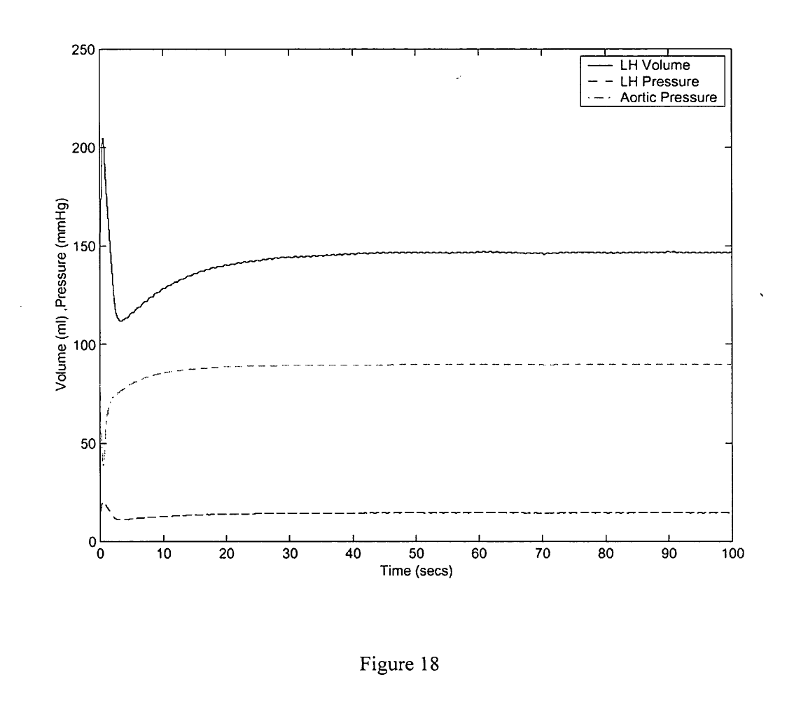

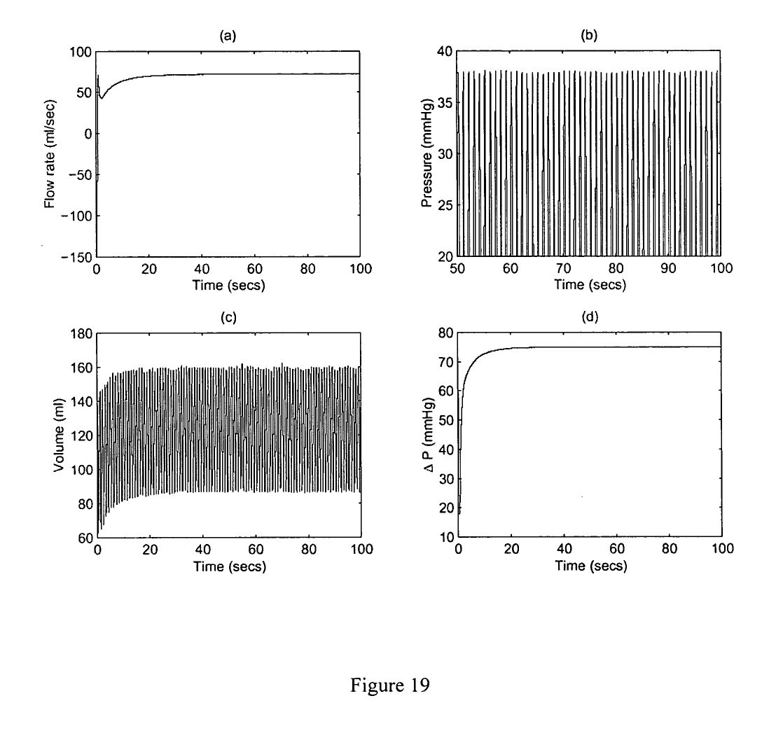

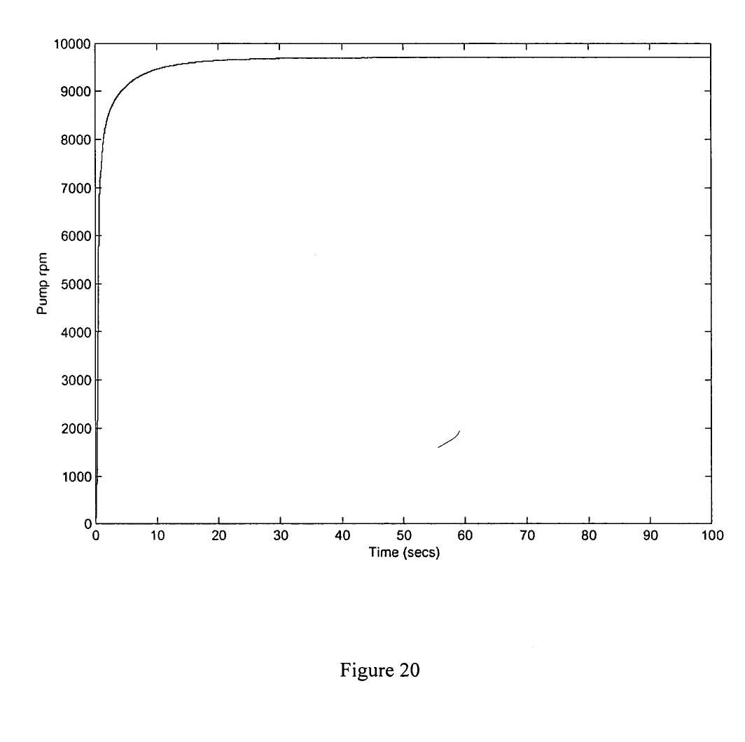

The LVAD with PI controller were finally tested for the case of an asystolic LH heart. FIGS. 18, 19, and 20 show the heart and VAD characteristics for an asystolic LH attached to a VAD. In FIGS. 18 and 19 the LH, and aorta volumes and pressures, pump flow rate, and pressure head settled to a single value after some initial oscillation. The RH volumes were stable and indicate adequate perfusion. The RH pressure stabilized at around 38/0, which is slightly elevated from the normal range despite a complete failure of the left heart. FIG. 20 shows that the rpm variations are absent as the asystolic LH does not produce any pressure variation.

Similar simulation studies were also performed for all three cases under heavy exercise. This was accomplished by reducing the time taken for each cardiac cycle and by altering the resistances for each block. The maximum factor by which the resistance was reduced was 3, as the pump flow rate exceeded 12 lpm, the design limit for most of the axial flow blood pumps.

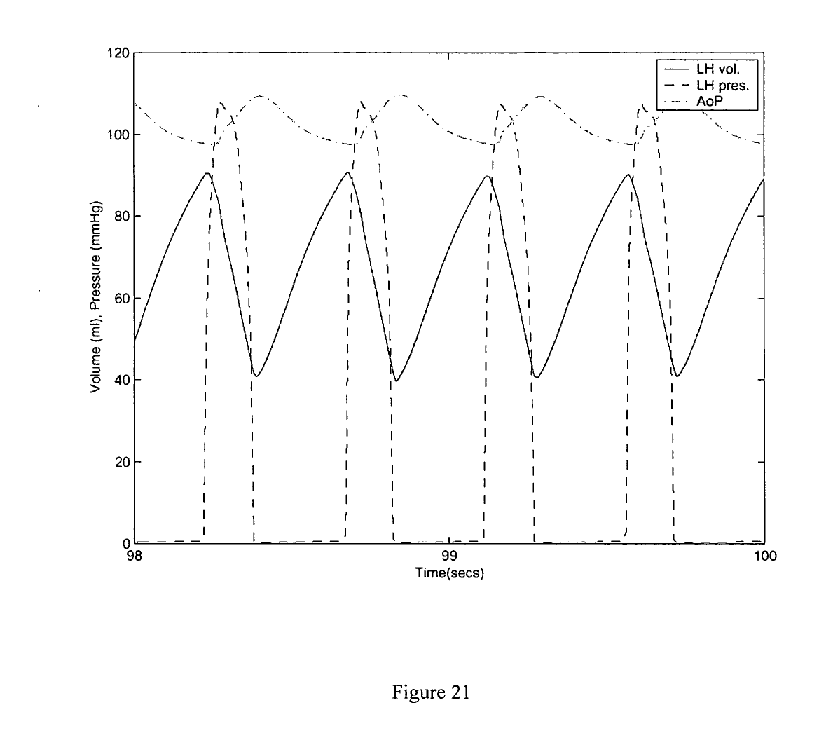

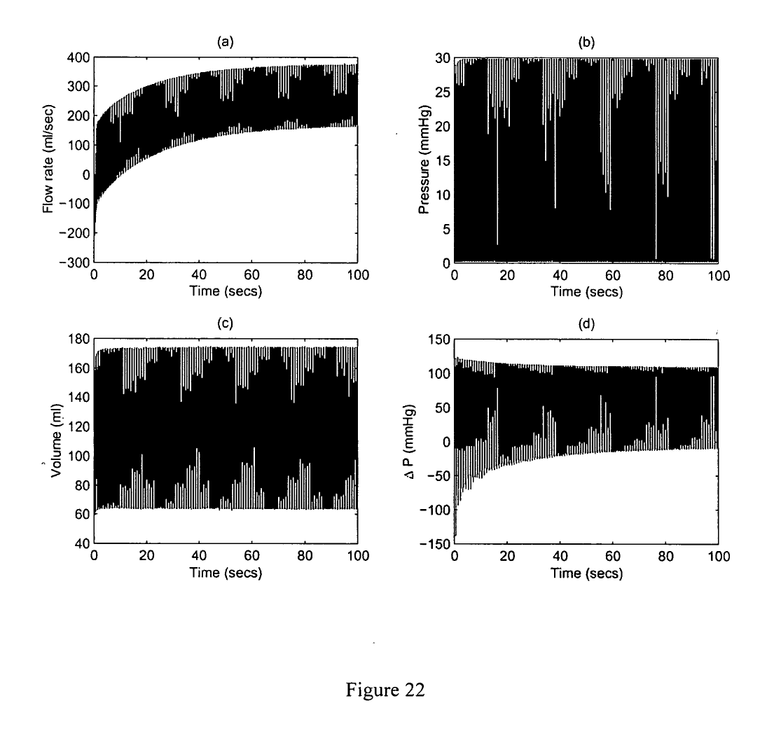

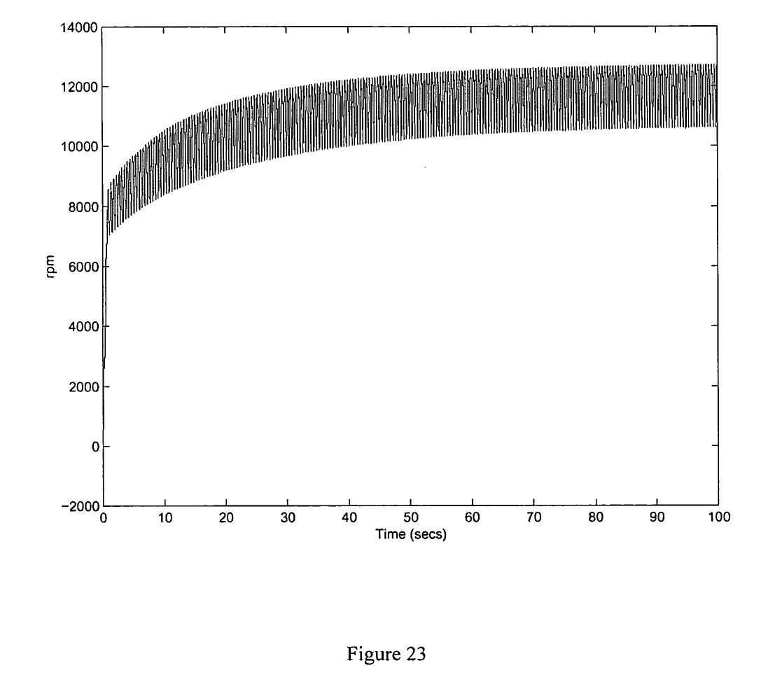

The cardiac demand during exercise was about triple the demand under rest. Shown in FIG. 21 are the LH characteristics and aortic pressure (AoP) for a healthy heart assisted by a VAD during exercise. The cardiac demand was about three times of what it would be under rest. The stroke volume is approximately 30 ml and a minimum LH volume of 50 ml gives an adequate safety margin against suction. The AoP remains approximately constant around 102 mmHg and the systolic pressure of the LH is 103 mmHg. The maximum RH pressure is 30 mmHg, which indicates healthy perfusion, FIG. 22b. The RH stroke volume is approximately 110 ml, FIG. 22c. The high initial pressure difference, FIG. 22d and backflow of blood, FIG. 22a is due to the zero rpm starting condition. The pump has some rpm variation even after reaching the limit cycle, FIG. 23, due to the pulsatility of the native heart.

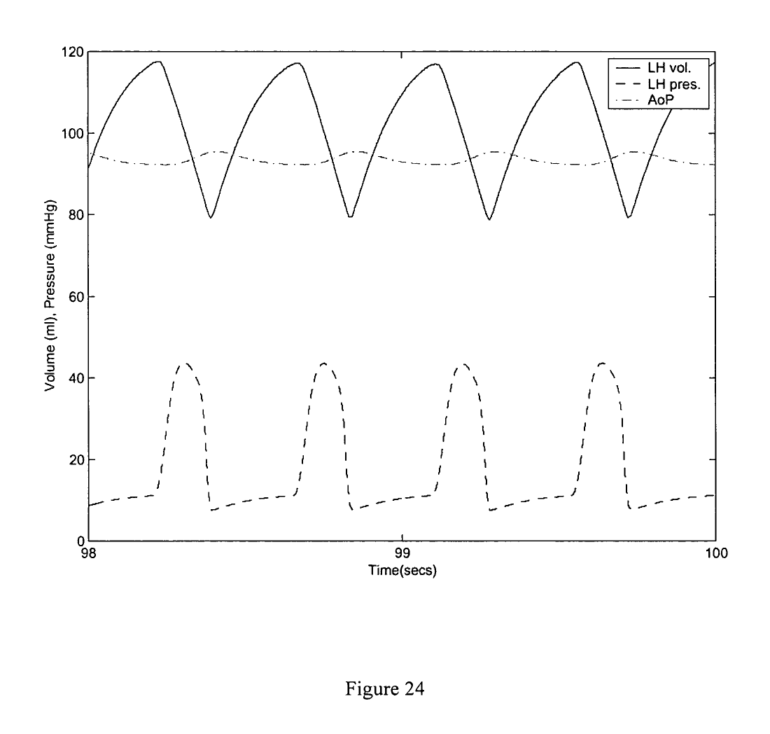

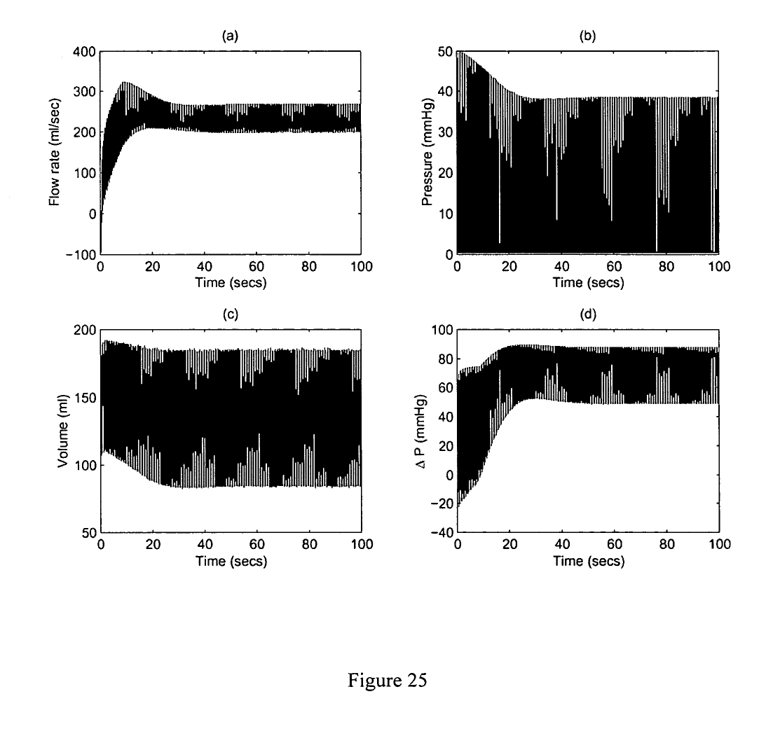

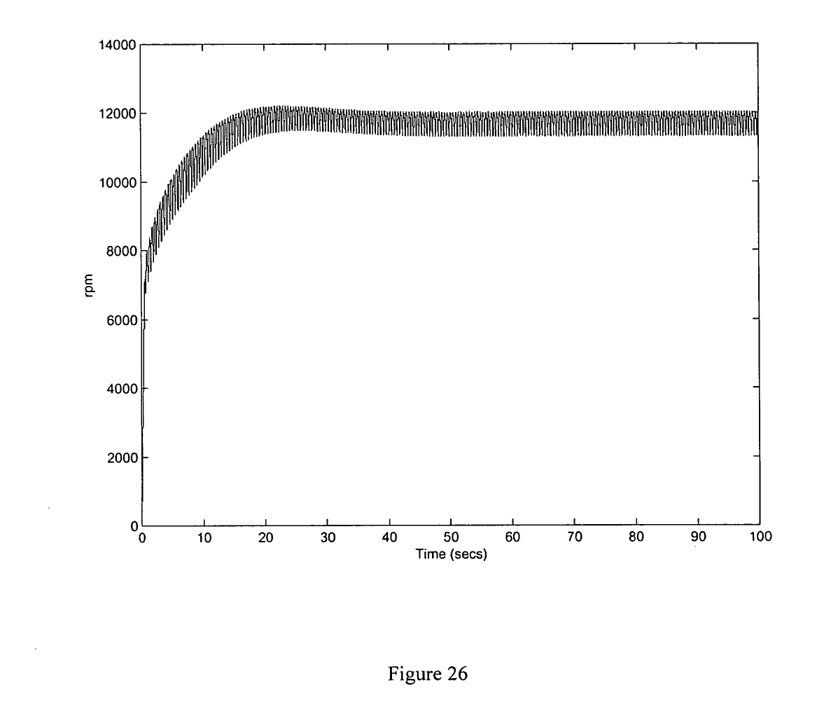

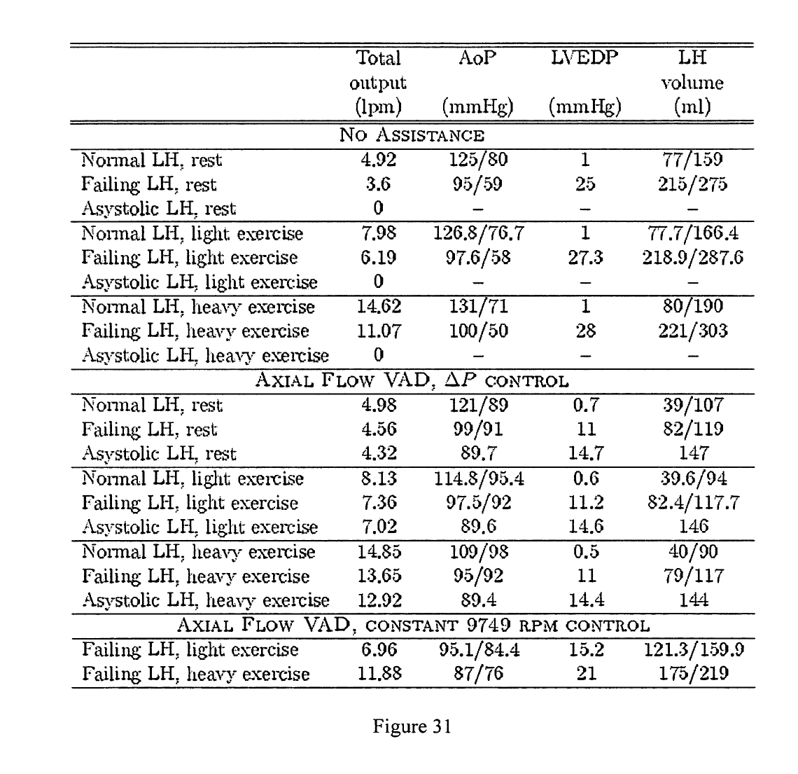

FIGS. 24, 25 and 26 show the results of the simulation for the weakened heart during exercise assisted by VAD with the designed controller. FIG. 24 indicates a fairly constant aortic pressure around 95 mmHg. The volume of the LH with VAD support reduces from 221/303 ml, observed without VAD assistance (FIG. 31), to approximately 85/120 ml, a normal range. The LH pressure is reduced to about 50/10 mmHg. The RH pressure reduces to 38/0 mmHg, FIG. 25b, which is slightly above the normal range, but is a significant improvement over a fatal 49/0 mmHg without the VAD 25b. FIG. 25a indicates no backflow through the pump and also that the average pressure head is closer to 75 mmHg setpoint, FIG. 25d, compared to a weakened heart without a VAD. It can be noted from FIG. 25c that the stroke volume increases from 80 ml, FIG. 25a, a failure condition, to 101 ml which is near the stroke volume for a normal heart under exercise, as seen from FIG. 25a. FIG. 26 shows that the rpm variations are reduced considerably at steady state and are less than the steady state rpm variation with healthy heart under exercise, FIG. 23.

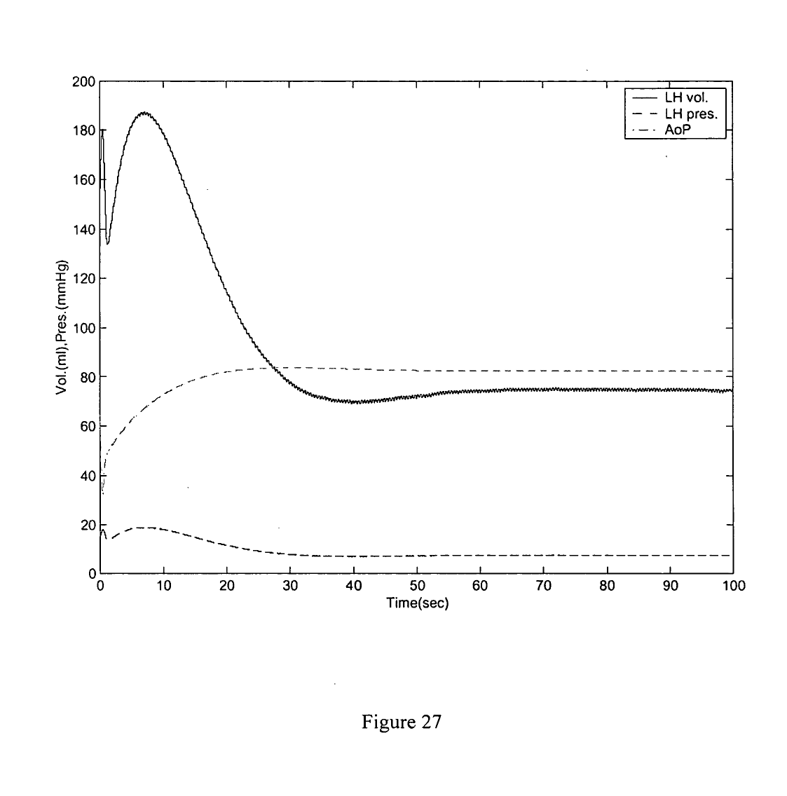

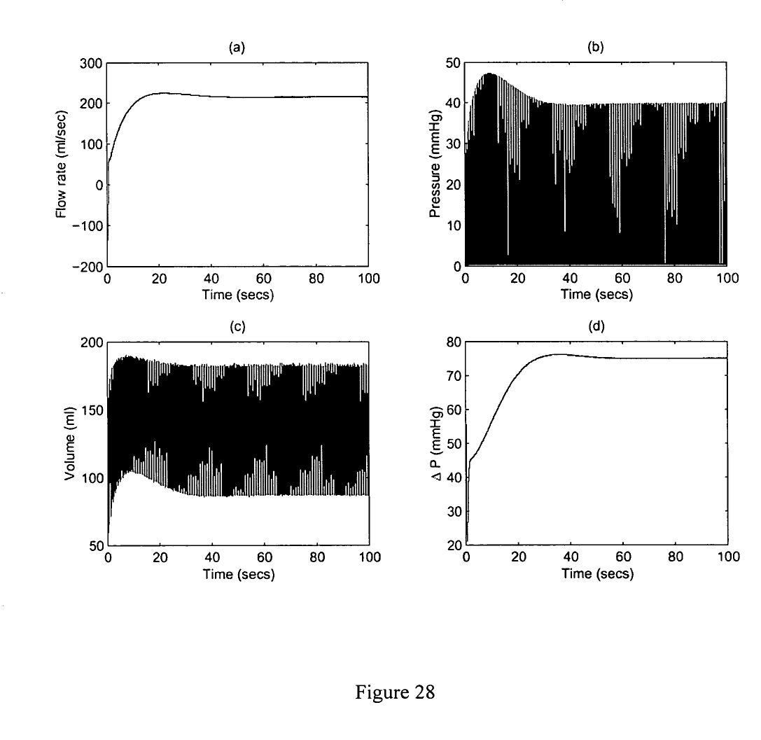

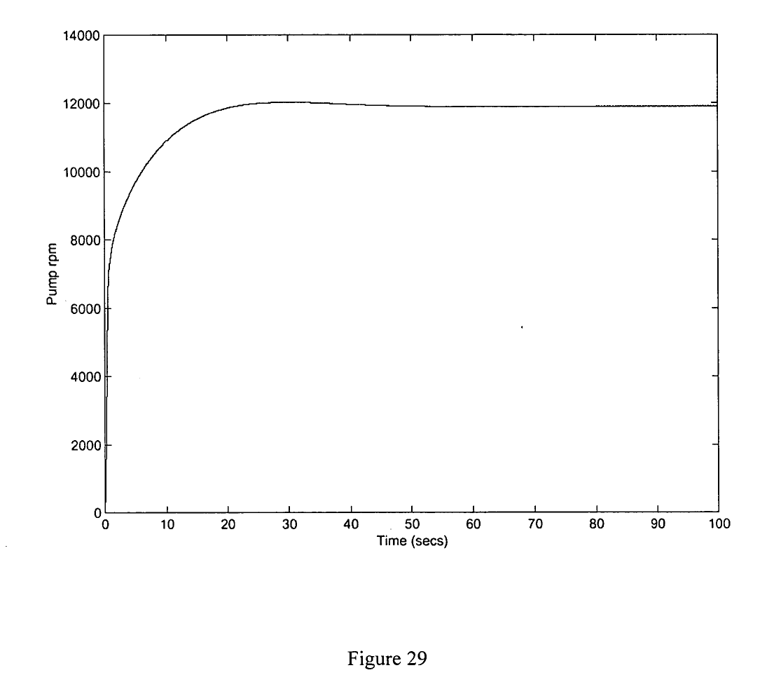

The LVAD with PI controller were finally tested under the case of an asystolic LH heart. FIGS. 27, 28 and 29 show the heart and VAD characteristics for an asystolic LH VAD assistance. In FIGS. 27 and 28 the LH, and aorta volumes and pressures, pump flow rate, and pressure head settled to a single value after some initial oscillation. The RH volumes were stable and indicate adequate perfusion. The RH pressure stabilized at around 38/0, which is slightly elevated from the normal range despite a complete failure of the left heart. FIG. 29 shows that the rpm variations are absent as the asystolic LH does not produce any pressure variation.

The comparison of rest and exercise cases shows that the control strategy of maintaining an average pressure difference, ΔP, leads to the correct adaptation to changing cardiac demand. This property of the proposed control strategy is in stark contrast to the performance of the traditional VAD controller, designed to maintain the pump rpm. Though some degree of physiological adaptation is achieved by maintaining a constant rpm, such a strategy is ineffective for the wide range of physical activities, and rapidly changing status of cardiac function (such as a sudden transition from failing to asystolic heart). With the constant rpm control strategy, broad range of physical and clinical conditions would require an external intervention to change the rpm setpoint according to some expert rule, model prediction or operator input. To illustrate this point, we consider the case of the VAD controller, which maintains a constant rpm. A constant rpm setpoint of 9749 rpm was selected for the axial flow pump, which were the average rpm observed with the designed ΔP controllers during rest. We, therefore, are testing the ability of the traditional control strategy to adequately perform during increased cardiac demand. The volume of the LH with VAD support at constant rpm reduces from 215/275 ml, observed without VAD assistance, FIG. 31 to approximately 175/219 ml with the axial flow pump. However, this value is still higher than the normal range. The RH pressure does not reduce significantly and is above the normal range, and is only marginally better than without VAD. The cardiac output is 11.88 lpm with the axial flow, while the normal heart during strenuous exercise generates 14.62 lpm. We can conclude that a constant rpm strategy is ineffective in adapting to changing demand, and requires an external intrusion to increase the rpm setpoint according to some expert rule or model prediction. On the other hand, the simulations show that the invented method of maintaining the desired average pressure differential leads to an adequate adaptation to changing cardiac demand in a completely autonomous way, and for a wide range of clinical conditions.

FIG. 31 compares cardiac output, AoP, the left ventricular end diastolic pressure (LVEDP) and LH volume for the conditions under which the Example was performed. FIG. 26 illustrates that the VAD reduced the LVEDP and increased the cardiac output to near normal values using the ΔP control strategy.

Thus, as described above, maintaining an average pressure difference by a PI controller between left heart and aorta provides an effective way to control the LVAD with the natural heart over a wide range of conditions. The PI controller offers a quick settling time and very low flow oscillations. This advantage is possible because maintaining the prescribed pressure differential synchronizes the assist and natural perfusion, thus indirectly incorporating natural cardiovascular regulation into the VAD control. The proposed control objective thus reflects the physiological demands of perfusion and is simple enough to allow for simple control laws, resulting in better device efficacy and reliability. The implementation of the invention requires either direct measurement of the pressure difference which controller will maintain, or estimation of the pressure difference, which, in this example, can be accomplished using the model of the VAD and readily available measurements of intrinsic pump parameters, such as electrical current, voltage and pump rpm.

### EXAMPLE 2

An adult mock circulation (consisting of a mock left ventricle, ventricular apical inflow cannulation and mock systemic vasculature with aortic root outflow cannulation) along with a centrifugal flow continuous blood pump (BioMedicus, Medtronic, Eden Prairie, Minn.) were used to test the viability of the ΔPa (maintaining an average pressure difference between the pulmonary vein and aorta) control strategy and compare it to constant rpm and constant pump pressure head (AP) control strategies.

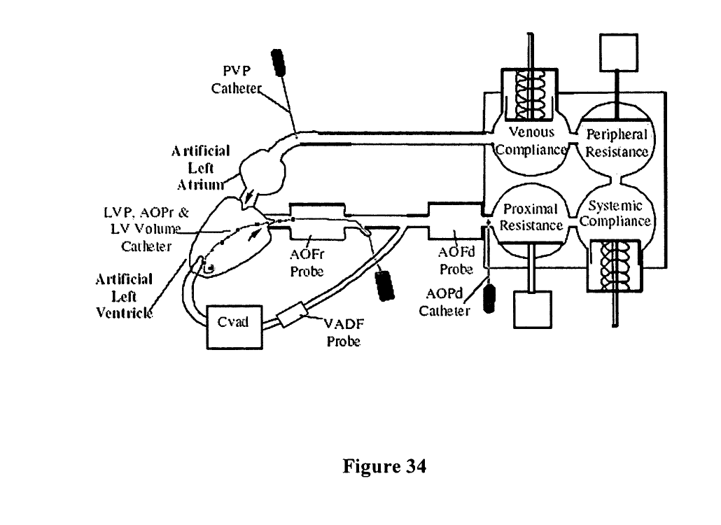

The adult mock circulation contained an atrium, ventricle, and systemic and coronary vasculature components as illustrated in FIG. 34. Based on a previous study, the adult mock circulation was shown to mimic human normal ventricle, failing ventricle, and partial cardiac recovery physiological responses as defined by characterizing hemodynamic parameters, ventricular pressure-volume relationship, aortic input impedance, and vascular mechanical properties. An artificial atrium, made of a flexible polymer sphere 50 mm in diameter, was connected upstream of the inflow valve of a mock ventricle. The mock ventricle contained a flexing, polymer sac inside a pressurization chamber. The ventricular sac was hemi-ellipsoid shaped and 70-mm wide at the base and 83-mm long from base to apex. The base was covered by a semi-rigid polymer dome 20-mm high, with mounts for inflow (mitral) and outflow (aortic) prosthetic valves. Metered pulses of compressed air (Utah Heart Controller, CardioWest, Tuscon, Ariz.) were delivered to the pressurization chamber during systole, compressing the ventricular sac to form coapting quadrants simulating contraction of the normal and dysfunctional ventricle and the delivery of cardiac stroke volume. An artificial aorta (polyurethane tube segment 25-mm diameter) was connected downstream of the outflow valve of the ventricular sac to the mock systemic vasculature. The mock systemic vasculature contained four integrated chambers that represent lumped proximal resistance, systemic compliance, peripheral resistance, and venous compliance. The introduction ports for the VAD uptake cannula were incorporated into the ventricular sac apex and VAD output flow cannula at the aortic root.

A high-fidelity pressure-volume conductance catheter (Millar Instruments, Houston, Tex.) was inserted into an aortic introducer port and passed retrograde through the aortic valve and down to the ventricular apex for simultaneous ventricular pressure, root aortic pressure and ventricular volume measurements. Single-tip high-fidelity catheters (Millar Instruments, Houston, Tex.) were inserted into introducer ports for measuring atrial pressure, distal aortic pressure, and drive-line pressures. Aortic root, aortic distal, and VAD output were measured with inline transit-time flow probes (Transonics, Ithaca, N.Y.). Pressure, flow, and volume transducers were pre- and post-calibrated, and transducer gains and offsets calculated and applied to ensure measurement accuracy. Gains were calculated for the LV volume data to match the stroke volume of the LV, as sensed by the aortic root flow probe. Offsets for the LV volume data were calculated taking into consideration the total flow and left ventricular end diastolic pressure (LVPed) data. Placement of instrumentation for hemodynamic measurements of pressures, flows, and volume is depicted in FIG. 34. Signal conditioning was accomplished using transducer amplifiers (Ectron, San Diego, Calif.), transit-time flow meters (Transonics, Ithaca, N.Y.), a volume conductance unit (Leycom, Sigma V, Netherlands), and other peripheral conditioners integrated in an instrumentation system compliant with Good Laboratory Practice (GLP) guidelines. Signal conditioned data were low-pass filtered at 60 Hz, analog-to-digitally converted (AT-MIO-16E-10 and LabVIEW, National Instruments) at a sampling rate of 400 Hz, and stored on a personal computer for post-processing and analysis.

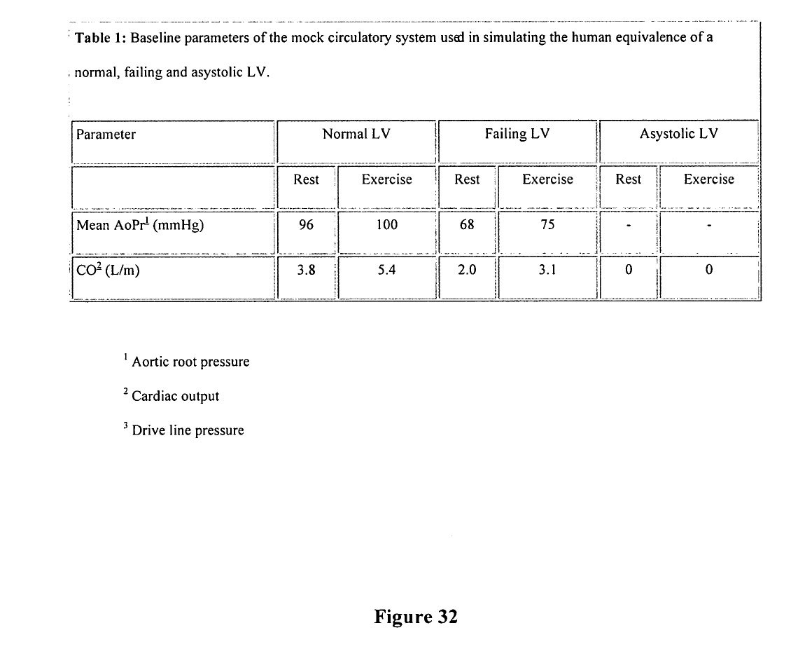

To study the range of applicability of the proposed approach, one normal and two different pathological cases of the VAD-assisted perfusion were simulated using this mock circulatory system. In the first case, a left ventricular assist device (LVAD) was attached to the human equivalent of a normal healthy heart (which is realistic when testing with animals or when the natural left heart (LH) function has completely recovered after VAD implantation). This equivalence included LVAD assistance of failing and asystolic left heart as depicted in Table 1 of FIG. 32.

Three scenarios were tested under rest and light exercise conditions. The heart rate was kept at 60 bpm during rest and at 100 bpm during light exercise. For all test conditions, 35% systole and 65% diastole were maintained. A lower value of the heart rate, and the resulting lower cardiac output during rest, were chosen to increase the variability in the cardiac demand as the maximum flow rate of the mock circulatory system is limited. The aortic input impedance and vascular mechanical properties were controlled to simulate the flow and impedance of the normal human vasculature. The vascular resistance (total peripheral resistance) and the driveline pressure (which controls the contractility of the LV) were adjusted to match the pressure and flow waveform characteristics of the human circulatory system under the described scenarios. Once the resistance and the driveline pressure were determined, they were used consistently to test the different control strategies.

For different clinical and cardiac demand conditions, the VAD rpm was adjusted manually until the setpoint for ΔPa, ΔP or rpm was reached. The setpoint for ΔPa was selected as the ΔPa value observed with normal unassisted heart (baseline case) at rest and was equal to approximately 95 mmHg. Based on the result of the previous simulation study, the setpoint for ΔP was selected as 75 mmHg. A pump speed of 1440 rpm (which was needed to restore the cardiac output to the physiologic level of 3.8 l/m for the case of failing heart at rest) was selected as the rpm setpoint. Once the setpoint was reached, the limit cycle hemodynamic waveforms were recorded with and without VAD assistance for each of the three control strategies. The characterizing hemodynamic parameters, waveform morphology, and ventricular pressure-volume loop responses were then calculated to identify differences in the performance with different control strategies for each test condition.

The differences in characterizing hemodynamic parameter values and ventricular pressure-volume loop response were calculated using a Hemodynamic Evaluation and Assessment Research Tool (HEART) program and supporting m-files developed in Matlab (MathWorks, Natick, Mass.). The pressure, flow, and volume waveforms were used to calculate the following hemodynamic parameters: mean pulmonary vein pressure; LVPED, VAD output flow; and the total flow. All hemodynamic parameters were calculated on a beat-to-beat basis, with all beats in each data set averaged to obtain a single representative mean value for each parameter. Pressure-volume loops were constructed by plotting ventricular pressure against ventricular volume, in which each loop represents one complete cardiac cycle (one beat). Characterizing hemodynamic parameters and pressure-volume loops were calculated for all experimental conditions.

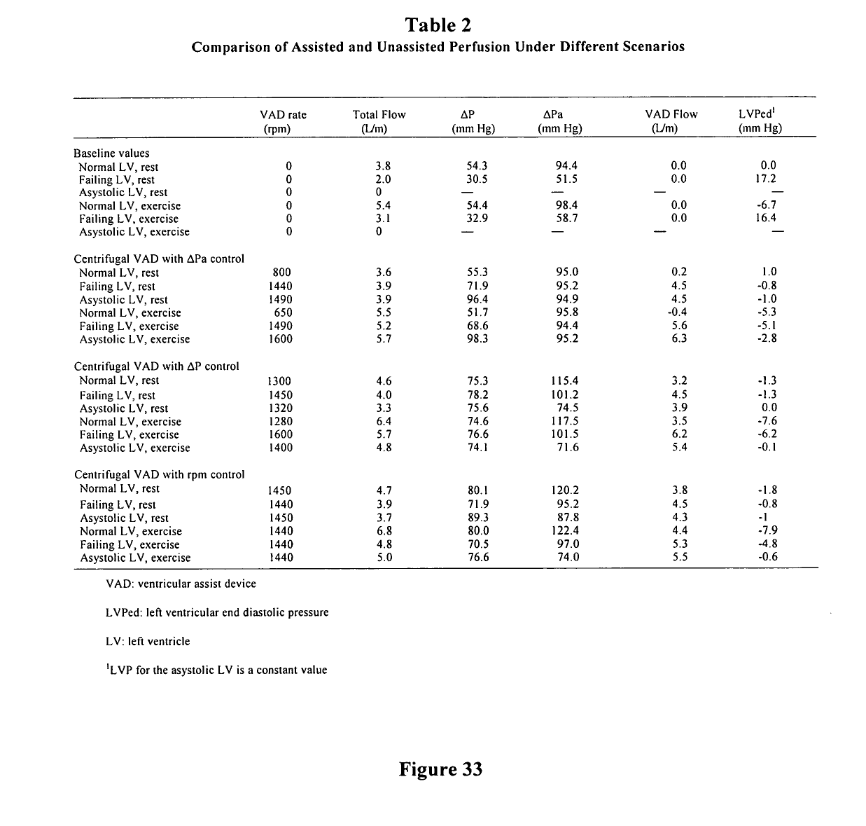

The hemodynamic parameters for a normal, failing and asystolic LV with and without continuous assist for each of the three control strategies during rest and light exercise are listed in Table 2 of FIG. 33. Without VAD assistance, the values of the total flow rate, ΔP and ΔPa decrease during ventricular failure at rest and exercise in comparison to the normal LV at rest and exercise. The left ventricular end diastolic pressure for all the control strategies remain within 3 mmHg of the baseline normal LV value. Since the left ventricular pressure and volume sensor was introduced through the aortic valve (as shown in FIG. 34), there is a back flow through that valve for baseline and all VAD assist scenarios. Table 2 indicates that all the tested control strategies increase the total flow, ΔP and ΔPa with failing and asystolic LV. Thus, the ΔPa control strategy maintains or restores the total flow rate to that of the physiologically normal heart during rest and exercise, and adapts best to the need for support. For example, in the case of the normal heart during rest and exercise, the average net VAD flow rate with this strategy is close to zero, as expected, since the native LV provides all the required cardiac output.

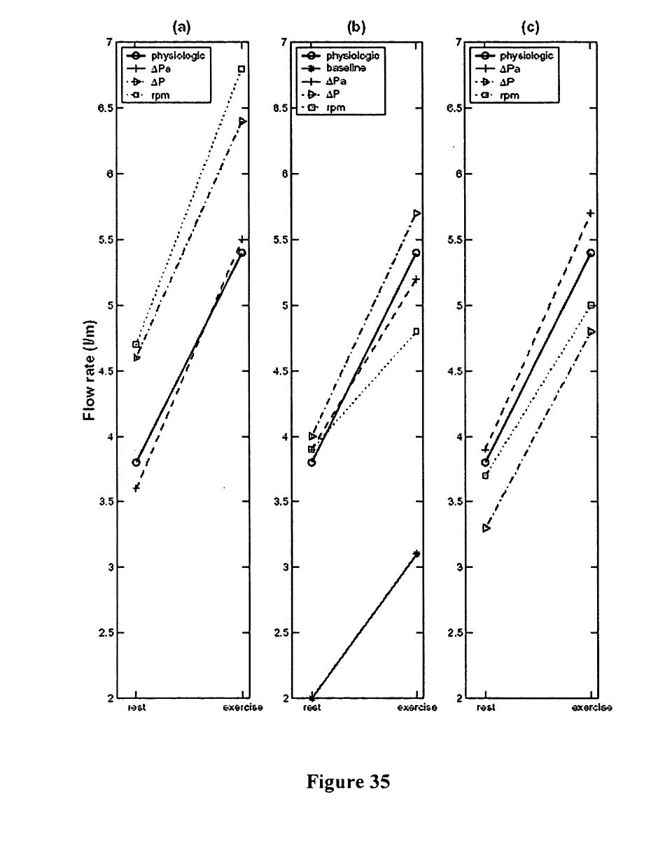

FIG. 35 compares the total flow rates (sum of VAD flow rate and cardiac output) at baseline and during assistance using constant rpm, ΔP and ΔPa control strategies during rest and exercise scenarios for each clinical test condition. The baseline cardiac output of 3.8 l/m at rest and 5.4 l/m during light exercise were considered to be physiological flows for the corresponding physical activities. The comparison of total flow rates produced with different control strategies showed that the ΔPa approach best matched the physiologic flow rate. The comparison of rest and exercise cases showed that the control strategy of maintaining an average ΔPa leads to the correct adaptation to changing cardiac demand.

For a normal LV, the ΔPa control strategy best matched the physiologic flow rates, FIG. 35a. At the same time, the constant rpm and ΔP control strategies resulted in higher than normal flow rate. The overpumping (highest when constant rpm is maintained) increased the risk of LV suction. FIG. 35b indicates that when the failing LV was assisted by a VAD, all three control strategies restored the total cardiac output to near physiologic level.

The ΔPa approach best matched the physiologic flow rate during rest and exercise. The ΔP approach leads to an output that was slightly higher than the physiologic flow rate. The constant rpm strategy resulted in a lower than normal flow rate during exercise, increasing the chances of under perfusion during higher cardiac demand. With an asystolic LV (FIG. 35c), the ΔPa strategy was the best approach at restoring the flow rates to near physiologic values, followed by constant rpm and ΔP strategies. Overall, the ΔPa strategy consistently produced a total flow rate that was the closest to the physiological flow rate for all heart conditions and physical activity scenarios.

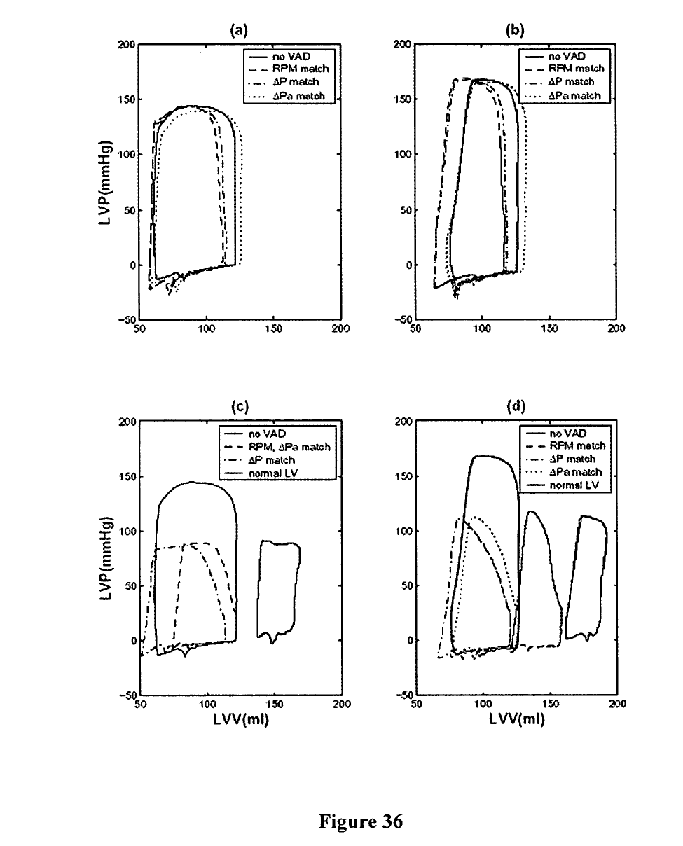

The left ventricular pressure-volume relationships for a normal and failing LV with and without assistance is shown in FIG. 36. All the tested control strategies caused a leftward shift in the pressure volume loop for a failing ventricle during rest and exercise, FIG. 36c&d, and a lowering of left ventricular end diastolic pressure, indicating a correct direction of adaptation for all the control strategies. Except for the result with ΔPa control, a leftward shift in the PV loop and lowering of LVPED was noticed for a normal heart assisted by a VAD during rest and exercise (FIG. 36a&b), indicating an increased likelihood for suction.

These results yield the following considerations. To begin with, adequate VAD control is important. Though the design of the VAD itself is important to the long-term success of the electromechanical implant, the control of the VAD determines the confidence of doctors and patients in the VAD as a permanent solution and an alternative to donor heart transplantation. The key requirement of the automatic control system is the adaptation of VAD-generated flow to the changing physiological requirements of the patient while reliably avoiding suction.

These in-vitro results show that maintaining a constant average ΔPa is an effective way to the correct adaptation of the cardiac output to changing requirements of the patient irrespective of the type of rotary pump used to assist perfusion. The physiological explanation of this conclusion rests with the fact that the vascular bed resistance can increase or decrease by a factor of 2 to 5 in response to the changing cardiac demand and is the dominant factor in regulating perfusion. The blood flow is inversely proportional to the vascular bed resistance so that maintaining a constant ΔPa with changing resistance can increase or decrease the flow rate by the same factor of 2 to 5.

The desired (reference) ΔPa can be maintained by adjusting the pump rpm within physiologically admissible limits despite the changing vascular resistance, stroke volume and heart rate, which represent the response of the natural regulatory mechanisms to the changing physiological cardiac output demand. The dominant role of the changing resistance in adaptation to physiological demand implies that by maintaining, on average, the prescribed ΔPa we, in effect, synchronize the assist and natural perfusion, thus indirectly incorporating natural cardiovascular regulation into VAD control.

The proposed approach to the control of rotary blood pump (RBP) requires that the natural regulatory mechanism functions properly in response to changing cardiac demand, which may not always be the case. For example, medical intervention may be necessary in the case of severe hypertension (often seen in the VAD recipients after initial recovery), which could lead to higher than normal arterial pressures, resulting in lung edema. Note that neither the alternative VAD control strategies, nor the natural heart can directly mitigate arterial hypertension, and the resulting lung edema. Consequently, the long-term goal may have to include the development of an automatic, autonomous and portable health monitoring and management system for patients with the permanent VAD or TAH, which would combine real time control of the blood pump with the automatic monitoring of the cardiac function, and, if necessary, emergency drug administration, and other advanced functionalities.

The primary advantage of the ΔPa control strategy is its ability to autonomously adjust the total output, defined as the sum of cardiac and pump outputs, to match the cardiac demand better than any alternative strategies. ΔPa, being the difference between the aortic and the pulmonary venous pressure (equal to left atrial pressure), is sensitive to changes both in preload and afterload. The current in-vitro study shows that the proposed strategy of maintaining the desired average ΔPa leads to an adequate adaptation for widely changing cardiac demand and clinical conditions in a completely autonomous way. The results show that, though some degree of physiological adaptation is achieved with constant rpm and constant ΔP, these alternatives are less effective for a wide range of physical activities, and rapidly changing status of cardiac function (such as a sudden transition from failing to asystolic heart). With the constant rpm control strategy, broad range of physical and clinical conditions would require an external intervention to change the rpm setpoint according to some expert rule, model prediction or operator input. The constant ΔP approach does not perform well for a broad range of clinical conditions of the native heart, which changed from normal to asystolic LV in this study, but adapts well to the changing cardiac demand due to different exercise levels. The in-vitro study is consistent with the results of computer simulations (presented in EXAMPLE 1), which showed that the ΔP control strategy adapts better to widely varying cardiac output requirements when compared to the traditional constant rpm control approach. Due to the limitations of the mock circulatory system, we were unable to test the higher cardiac demand conditions to make an in-vitro comparison between the different control strategies. In the limited range of cardiac demands that could be tested in-vitro, the performance of the ΔP control strategy is superior to ΔP and constant rpm control alternatives.

Using the proposed approach, both the natural heart and the assist device are contributing to the pumping action of maintaining an average ΔPa. If cardiac function improves, the native heart will increase its contribution to maintaining the reference pressure difference, with the VAD controller autonomously and automatically responding to the decreased need for assisted perfusion, as evidenced by the near zero net VAD flow rate with the normal heart during rest and exercise. When the net flow rate through the VAD is close to zero, blood does not stagnate inside the VAD, though the residence time of blood in the pump is higher, increasing the probability of hemolysis. Since this scenario occurs only with a normal or near normal heart, the patient could be weaned from the pump at this stage.

The ability of the proposed control strategy to automatically adjust its contribution towards maintaining ΔPa may prove to be well suited to the application of the ventricular assist devices in cardiac recovery therapy of end stage heart failure (alone or in combination therapy), as well as in the destination therapy.

Though not directly addressed in this paper, the over-arching principle behind the proposed approach of maintaining key pressure differences with mechanical blood pumps, while relying on the natural regulation to adjust the resistances to the blood flow to meet the physiological demand is also applicable to the case of pulsatile ventricular assist devices, as well as the total artificial heart. In the case of the TAH, the blood pump should be controlled to maintain key pressure differences at the average reference values, which, in the case of pulmonary circulation, is the difference between pulmonary arterial and vena cava pressures.

In its current form, for the case of VAD control the proposed approach requires two pressure sensors, which may not be clinically feasible for long term implantation. However, for different types of blood pumps, it may be possible to estimate ΔPa using the pump model, and only intrinsic and readily measurable pump parameters (such as pump rpm, voltage and current), eliminating the need for implantable pressure sensors. The approach which utilizes a blood pump as both the actuator and the flow or pressure sensor, can be viewed as a “sensorless” control. Sensorless estimation of ΔPa is currently being pursued as a follow-up investigation.

These results also show that maintaining an average pressure difference between the pulmonary vein and aorta (ΔPa) provides an effective way to control a continuous flow LVAD over a wide range of physiological and cardiac demand conditions while reducing the probability of suction. Change in vascular resistance is the dominant regulatory mechanism in meeting the physiological requirements for blood perfusion. Maintaining the desired average ΔPa by adjusting the pump rpm during changing cardiac demand, in effect, synchronizes the assist and natural perfusion. Therefore, the proposed control strategy indirectly incorporates natural cardiovascular regulation, which changes vascular resistance, into VAD control. The comparison with the VAD control systems, which maintain either constant reference pump rpm, or constant pump pressure head (AP) shows that the proposed approach is superior in autonomously maintaining an adequate perfusion during changing cardiac demand for the test conditions simulated in vitro. Since the ΔPa control strategy automatically adjusts its contribution to the total flow based on the function of the native ventricle, the proposed approach may prove to be well suited to the application of the ventricular assist devices in recovery therapy.

Having described these aspects of the invention, it is understood that the invention defined by the appended claims is not to be limited by particular details set forth in the above description, as many apparent variations thereof are possible without departing from the spirit or scope thereof.

1. A method for flowing a body fluid through a portion of the body, comprising:
providing a pump for the body fluid;
determining a first pressure differential across the pump;
estimating the second pressure differential across a portion of the body; and
maintaining the second pressure differential at a substantially constant value by regulating the first pressure differential.

2. The method of claim 1, further comprising determining the first pressure differential by measuring the pressure at the input and output of the pump using pressure sensors.

3. The method of claim 1, further comprising determining the first pressure differential by estimating the input and output pressure of the pump using the operating parameters of the pump.

4. The method of claim 1, including regulating the first pressure differential using a control system.

5. The method of claim 4, wherein the control system contains a feedback controller for maintaining the first pressure differential.

6. The method of claim 1, wherein the body fluid is blood and the body portion comprises a part of the circulatory system.

7. The method of claim 6, wherein the body portion comprises a part of the heart.

8. The method of claim 7, wherein the body portion comprises the left ventricle and the aorta, or the pulmonary vein, atrium, and the aorta.

9. A method for controlling the pressure differential across a portion of the body, comprising:
providing a pump for fluid flowing through a portion of the body;
determining a first pressure differential across the pump;
estimating the second pressure differential across the body portion; and
maintaining the second pressure differential at a substantially constant value by regulating the first pressure differential.

10. The method of claim 9, further comprising determining the first pressure differential by measuring the pressure at the input and output of the pump using pressure sensors.

11. The method of claim 9, further comprising determining the first pressure differential by estimating the input and output pressure of the pump using the operating parameters of the pump.

12. The method of claim 9, including regulating the first pressure differential using a control system.

13. The method of claim 12, wherein the control system contains a feedback controller for maintaining the first pressure differential.

14. The method of claim 9, wherein the body fluid is blood and the body portion comprises a part of the circulatory system.

15. The method of claim 14, wherein the body portion comprises a part of the heart.

16. The method of claim 15, wherein the body portion comprises the left ventricle and the aorta, or the pulmonary vein, atrium, and the aorta.

17. A method for flowing a body fluid through a portion of the body, comprising:
providing a pump for the fluid;
determining a model for the circulation of the fluid through a portion of the body;
designing a controller for the pressure differential across the pump based on the model; and
using the controller to maintain the pressure differential at a substantially constant value.

18. The method of claim 17, wherein the model represents the body portion as a fixed number of blocks having a resistance to and storage of the fluid.

19. The method of claim 18, wherein the blocks have a compliance, pressure, and volume for the fluid.

20. The method of claim 17, wherein the controller is a feedback controller.

21. The method of claim 17, wherein the pressure differential is maintained at a substantially constant value either instantaneously or as an average value.

22. A method for controlling the pressure differential across a portion of the body, comprising:
providing a pump for a fluid flowing through a portion of the body;
determining a model for the circulation of the fluid;
designing a controller for the pressure differential across the pump based on the model; and
using the controller to maintain the pressure differential at a substantially constant value.

23. A system for pumping a body fluid through a portion of the body, comprising:
a pump;
a model for estimating the circulation of a body fluid through a portion of the body; and
a controller for regulating the pressure differential across the pump based on the model.

24. The system of claim 23, wherein the model represents the body portion as a fixed number of blocks having a resistance to and storage of the fluid.

25. The system of claim 25, wherein the blocks have a compliance, pressure, and volume for the fluid.

26. The system of claim 23, wherein the controller is a feedback controller.

27. The system of claim 23, wherein the body fluid is blood and the pump is a ventricular assist device.

27. A method for controlling the flow of a body fluid, comprising:
providing a pump in the flow of the body fluid;
determining a model for the circulation of the body fluid through a portion of the body;
designing a controller for the pressure differential across the pump based on the model; and
using the controller to maintain the pressure differential at a substantially constant value.

28. A method for flowing a body fluid through a portion of the body, comprising:
providing a pump for the body fluid;
determining a first pressure differential across a portion of the body;
estimating the second pressure differential across a portion of the body; and
maintaining the second pressure differential at a substantially constant value by regulating the first pressure differential.

29. The method of claim 28, further comprising determining the first pressure differential by measuring the pressure across the portion of the body using pressure sensors.

30. The method of claim 28, further comprising determining the first pressure differential by estimating the pressure differential using the operating parameters of the pump.

31. A method for controlling the pressure differential across a portion of the body, comprising:
providing a pump for fluid flowing through a portion of the body;
determining a first pressure differential across the pump;
estimating or measuring a second pressure differential across the body portion; and
maintaining the second pressure differential at a substantially constant value by regulating the first pressure differential.

32. The method of claim 31, further comprising determining the first pressure differential by measuring the pressure across the portion of the body using pressure sensors.

33. The method of claim 31, further comprising determining the first pressure differential by estimating the pressure differential using the operating parameters of the pump.

34. A method for flowing a fluid through a portion of the body, comprising:
providing a pump for the fluid;
determining a model for the circulation of the fluid through a portion of the body;
designing a controller that utilizes the model to control the pressure differential; and
using the controller to maintain the pressure differential at a substantially constant value.

35. The method of claim 34, wherein the pressure differential is maintained at a substantially constant value either instantaneously or as an average value.

36. The method of claim 22, wherein the pressure differential is maintained at a substantially constant value either instantaneously or as an average value.
